(19) United States
(12) Patent Application Publication
(10) Pub. No.: US 2020/0087516 A1
(43) Pub. Date: Mar. 19, 2020

(54) RED FLUORESCENT PROBE FOR USE IN DETECTION OF PEPTIDASE ACTIVITY

(71) Applicant: The University of Tokyo, Tokyo (JP)

(72) Inventors: Yasuteru URANO, Tokyo (JP); Mako KAMIYA, Tokyo (JP); Ryo TACHIBANA, Tokyo (JP)

(21) Appl. No.: 16/485,962

(22) PCT Filed: Feb. 16, 2018
(86) PCT No.: PCT/JP2018/005515

§ 371 Date: Aug. 14, 2019

Related U.S. Application Data
(60) Provisional application No. 62/460,557, filed on Feb. 17, 2017.

(51) Int. Cl. C09B 11/24; C12Q 1/37; A61K 49/00

(52) U.S. Cl. CPC C09B 11/24; A61K 49/0023; A61K 49/0008; C12Q 1/37

(57) ABSTRACT

[Problem]

A problem addressed by the present invention is to provide a novel fluorescent probe having excellent tissue permeability that is capable of detecting the peptidase activity expressed at a high level in cancer cells and the like as a response of long-wavelength red fluorescence.

[Solution]

A compound represented by formula (I) or a salt thereof:

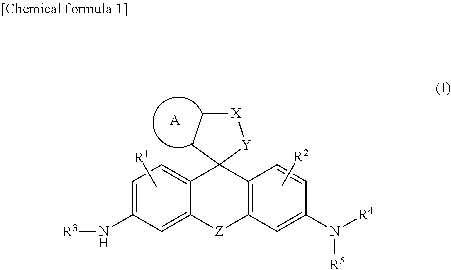

[In the formula, A represents a ring structure selected from the group consisting of a thiophene ring, a cyclopentene ring, a cyclopentadiene ring, and a furan ring;
X represents a C₀-C₃ alkylene group;
Y represents O, S, C(═O)O, or NH,
Z represents O, C(R^{a}) (R^{b}), Si(R^{a}) (R^{b}), Ge(R^{a}) (R^{b}), Sn(R^{a}) (R^{b}), Se, P(R^{c}), or P(R^{c}) (═O) (where R^{a} and R^{b} each independently represent a hydrogen atom or an alkyl group, and R^{c} represents a hydrogen atom, an alkyl group, or an aryl group);
R¹ and R² each independently represent from one to three of the same or different substituents selected from the group consisting of a hydrogen atom, a hydroxyl group, a halogen atom, and an alkyl group, a sulfo group, a carboxyl group, an ester group, an amide group, and an azide group each of which may be substituted;
R³ represents an acyl residue derived from an amino acid (where the acyl residue is a residue obtained by removing an OH group from a carboxyl group of the amino acid);
R⁴ and R⁵ each independently represent a hydrogen atom or an alkyl group (where when R⁴ or R⁵ is an alkyl group, the R⁴ or R⁵, together with R², may form a ring structure comprising a nitrogen atom to which R⁴ and R⁵ are bonded).]

### TECHNICAL FIELD

The present invention relates to a fluorescent probe for detection of peptidase activity. More specifically, the present invention relates to a novel fluorescent probe capable of detecting peptidase activity such as aminopeptidase by fluorescence in the red region, and to a detection method and device using said fluorescent probe.

### BACKGROUND ART

With the number of cancer patients and deaths increasing year by year, the development of treatment methods continues to be anticipated. The single most reliable cancer treatment method at the present time is the early detection and reliable surgical removal of the cancer, but it is difficult to completely remove cancer tissue that is difficult to see completely, leading to recurrence.

On the other hand, enhanced expression of γ-glutamyl transferase (GGT), which is a peptidase (protease), has been observed in cancer cells, and this enhanced expression is reported to be related to drug resistance. The detection of γ-glutamyl transferase can therefore be expected to lead to the development of a diagnostic method for identifying cancer cells and cancer tissue at high accuracy.

The present inventors previously developed a fluorescent probe capable of detecting the activity of γ-glutamyl transferase based on a fluorescent dye which exhibits intramolecular spirocyclization equilibrium (Non-Patent Reference 1, etc.)

However, for the absorption and emission wavelengths of such conventional fluorescent probes, the fluorescence wavelength is 550 nm or less (green fluorescence) which, although capable of detecting cancer cells and the like present on a tissue surface at high sensitivity, imposed a limitation in that the probes could not be applied to cancer cells present below living tissues or within organs such as lymph node metastases.

### PRIOR ART REFERENCES

### Non-Patent References

Non-Patent Reference 1: Y. Urano et al., Sci. Transl. Med., 2011, Vol. 3, pp. 110ra119

### SUMMARY OF THE INVENTION

### Problems to be Solved by the Invention

Therefore, a problem addressed by the present invention is to provide a novel fluorescent probe having excellent tissue permeability that is capable of detecting the peptidase activity expressed at a high level in cancer cells and the like as a response of a long-wavelength red fluorescence. Another problem addressed is to permit multicolor imaging by using such a red fluorescent probe in combination with a conventional green fluorescent probe and to provide a system capable of visualizing and detecting cancer cells accurately and at high sensitivity.

As a result of in-depth studies intended to solve the above problems, the present inventors discovered that a fluorescent probe which is colorless and nonfluorescent before contact with a target peptidase but shows a response of red fluorescence of near 600 nm upon reaction with said peptidase is obtained by using a compound structured such that a group cleaved by a peptidase is introduced into a rhodamine skeleton linked with a thiophene ring or the like and optimizing the intramolecular spirocyclization characteristics, and thereby perfected the present invention.

Specifically, according to an aspect of the present invention, there are provided:

<1> A compound represented by the following formula (I) or a salt thereof.

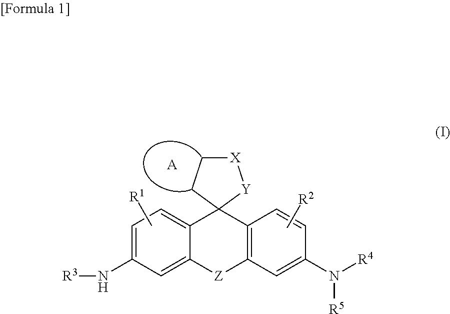

[In the formula, A represents a ring structure selected from the group consisting of a thiophene ring, a cyclopentene ring, a cyclopentadiene ring, and a furan ring;

X represents a C₀-C₃ alkylene group;
Y represents O, S, C(═O)O, or NH,
Z represents O, C(R^{a}) (R^{b}), Si(R^{a}) (R^{b}), Ge(R^{a}) (R^{b}), Sn(R^{a}) (R^{b}), Se, P(R^{C}), or P(R^{c}) (═O) (where R^{a} and R^{b} each independently represent a hydrogen atom or an alkyl group, and R^{c} represents a hydrogen atom, an alkyl group, or an aryl group);
R¹ and R² each independently represent from one to three of the same or different substituents selected from the group consisting of a hydrogen atom, a hydroxyl group, a halogen atom, and an alkyl group, sulfo group, carboxyl group, ester group, amide group, and azide group each of which may be substituted;
R³ represents an acyl residue derived from an amino acid (where the acyl residue is a residue obtained by removing an OH group from a carboxyl group of the amino acid);
R⁴ and R⁵ each independently represent a hydrogen atom or an alkyl group (where when R⁴ or R⁵ is an alkyl group, the R⁴ or R⁵, together with R², may form a ring structure comprising a nitrogen atom to which R⁴ and R⁵ are bonded).];
<2> The compound or salt thereof according to <1>, wherein A is a thiophene ring;
<3> The compound or salt thereof according to <1>, wherein Y is O;
<4> The compound or salt thereof according to <1>, wherein Z is Si(R^{a}) (R^{b}) or C(R^{a}) (R^{b});
<5> The compound or salt thereof according to <1>, wherein R³ is a glutamic acid residue;
<6> The compound or salt thereof according to <1>, wherein R¹, R², R⁴, and R⁵ are all hydrogen atoms; and
<7> The compound or salt thereof according to <1>, wherein the compound represented by formula (I) is a compound selected from the group shown below;

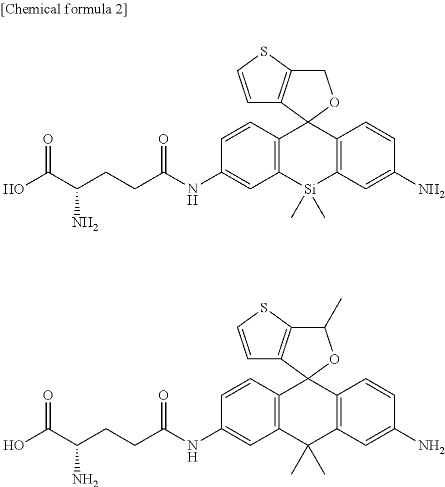 [SMILES: C[Si]1(C)c2cc(N)ccc2C2(OCc3sccc32)c2ccc(NC(=O)CC[C@H](N)C(=O)O)cc21 ; CC1OC2(c3ccc(N)cc3C(C)(C)c3cc(NC(=O)CC[C@H](N)C(=O)O)ccc32)c2ccsc21]

In another embodiment, the present invention provides:

<8> A fluorescent probe for detection of peptidase activity comprising a compound or salt thereof according to any of <1>-<7>;
<9> A kit for detecting or for visualizing a target cell that expresses a specific peptidase comprising the fluorescent probe for detection of peptidase activity according to <8>;
<10> The kit according to <9>, wherein the peptidase is γ-glutamyl transpeptidase, dipeptidyl peptidase IV(DPP-IV), or calpain; and
<11> The kit according to <9>, wherein the target cell is a cancer cell.

In a further embodiment, the present invention also relates to a method for detecting or visualizing a target cell that expresses a specific peptidase, specifically:

<12> A method for detecting or visualizing a target cell that expresses a specific peptidase using a compound or salt thereof according to any of <1>-<7>;
<13> The method according to <12>, characterized by comprising a step for bringing the compound or salt thereof into contact with the target cell ex vivo; and a step for observing a fluorescence response due to a reaction between a peptidase specifically expressed in the target cell and the compound or salt thereof;
<14> The method according to <13>, comprising observing the fluorescence response using a fluorescence imaging means;
<15> The method according to <12>, wherein the peptidase is γ-glutamyl transpeptidase, dipeptidyl peptidase IV (DPP-IV), or calpain;
<16> The method according to <12>, wherein the target cell is a cancer cell; and
<17> The use of a compound or salt thereof according to any of <1>-<7> for detecting or for visualizing a target cell that expresses a specific peptidase;

In a further embodiment, the present invention also relates to a device equipped with a means for observing a fluorescence response by the fluorescent probe for detection of peptidase activity; specifically, the present invention provides:

<18> A device equipped with a fluorescence imaging means for observing a fluorescence response due to a reaction between a peptidase specifically expressed in a target cell and a compound or a salt thereof according any of <1>-<7>; and
<19> The device according to <18>, wherein the device is an endoscope or an in vivo fluorescence imaging device.

The fluorescent probe of the present invention is colorless and nonfluorescent before contact with a target peptidase, but permits a fluorescence response in the red region to be detected due to reaction with the peptidase specifically and in an on/off manner.

Also, using the red fluorescent probe of the present invention in combination with a conventional green fluorescent probe enables multicolor imaging using a plurality of fluorescence response regions and also makes it possible to visualize and detect cancer cells and the like accurately and at high sensitivity.

### BRIEF DESCRIPTION OF THE DRAWINGS

FIG. 1 is an intramolecular equilibrium/kinetic model of a compound having a rhodamine skeleton.

FIG. 2 is a graph showing the changes in the absorption spectra of fluorescent probe 1 (gGLu-MHM4ThPCR550) of the present invention and MHM4ThPCR550 having no gGlu group as a comparison.

FIG. 3 is a graph showing the changes in the fluorescence spectra of fluorescent probe 1 (gGLu-MHM4ThPCR550) of the present invention and MHM4ThPCR550 having no gGlu group as a comparison.

FIG. 4 is a graph showing changes in the absorption spectrum of fluorescent probe 2 (gGlu-HM3ThPSiR600) of the present invention. Also shown are the absorption spectra of HM3ThPSiR600 having no gGlu group and HM3ThPAcSiR600 having an Ac group instead of a gGlu group as a comparison.

FIG. 5 is a graph showing the changes over time in the fluorescence intensity when γ-glutamyl transpeptidase (GGT) was added to fluorescent probe 1 (gGlu-MHM4ThPCR550) of the present invention.

FIG. 6 is a graph showing the changes over time in the fluorescence intensity when γ-glutamyl transpeptidase (GGT) was added to fluorescent probe 2 (gGlu-HM3ThPSiR600) of the present invention.

FIG. 7 shows in vivo imaging images of a cancer peritoneal dissemination model mouse taken using fluorescent probe 1 (gGlu-MHM4ThPCR550) of the present invention; and

FIG. 8 shows in vivo imaging images of a cancer peritoneal dissemination model mouse taken using fluorescent probe 2 (gGlu-HM3ThPSiR600) of the present invention.

### BEST MODE FOR CARRYING OUT THE INVENTION

Embodiments of the present invention are described below. The scope of the present invention is not limited to the above described embodiments, and modifications other than those of the examples described below may be made, as appropriate, insofar as the intent of the present invention is not compromised.

### 1. Definitions

In the present specification, “halogen atom” means a fluorine atom, chlorine atom, bromine atom, or iodine atom.

In the present specification, “alkyl” may be an aliphatic hydrocarbon group in a linear, branched, or cyclic configuration, or any combination thereof. The number of carbon atoms in the alkyl group is not particularly restricted, but is, for example, 1-20 (C₁₋₂₀), 1-15 (C₁₋₁₅), or 1-10 (C₁₋₁₀) When a number of carbon atoms is specified, it means an “alkyl” having a number of carbon atoms within that numerical range. For example, C₁₋₈ alkyls include methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. In the present specification, an alkyl group may have one or more optional substituents. Examples of substituents include, but are not limited to, an alkoxy group, halogen atom, amino group, mono- or di-substituted amino group, substituted silyl group, or acyl, or the like. When an alkyl group has two or more substituents, they may be the same or different. The same is also true for the alkyl moiety of other substituents (for example, an alkoxy group, arylalkyl group, or the like) comprising an alkyl moiety.

In the present specification, when certain functional groups are defined as “optionally substituted,” the type of substituent, substitution position, and number of substituents are not particularly restricted. When there are two or more substituents, they may be the same or different. Examples of substituents include, but are not limited to, an alkyl group, alkoxy group, hydroxyl group, carboxyl group, halogen atom, sulfo group, amino group, alkoxycarbonyl group, oxo group, or the like. Other substituents may be present in these substituents. Examples of such cases include, but are not limited to, an alkyl halide group, dialkylamino group, or the like.

In the present specification, “alkenyl” means a linear or branched hydrocarbon group having at least one carbon-carbon double bond. Non-limiting examples include vinyl, allyl, 1-propenyl, isopropenyl, 1-butenyl, 2-butenyl, 3-butenyl, 1,3-butanedienyl, 1-pentenyl, 2-pentenyl, 3-pentenyl, 4-pentenyl, 1,3-pentanedienyl, 1-hexenyl, 2-hexenyl, 3-hexenyl, 4-hexenyl, 5-hexenyl, and 1,4-hexanedienyl. The double bond may have either a cis conformation or trans conformation.

In the present specification, “alkynyl” means a linear or branched hydrocarbon group having at least one carbon-carbon triple bond. Non-limiting examples include ethynyl, propynyl, 2-butynyl, and 3-methylbutynyl.

In the present specification, “cycloalkyl” means a monocyclic or polycyclic non-aromatic ring system composed of the above alkyls. This cycloalkyl can be unsubstituted or substituted by one or more substituents which may be the same or different. Non-limiting examples of monocyclic cycloalkyls include cyclopropyl, cyclopentyl, cyclohexyl, and cyclopentyl. Non-limiting examples of polycyclic cycloalkyls include 1-decalinyl, 2-decalinyl, norbornyl, adamantyl, and the like. This cycloalkyl may also be a heterocycloalkyl including one or more hetero atoms (for example, an oxygen atom, nitrogen atom, or sulfur atom) as ring constituent atoms. Any —NH in the heterocycloalkyl ring may be protected, for example, as an —N(Boc) group, —N(CBz) group, or —N(Tos) group, and nitrogen atoms or sulfur atoms in the ring may be oxidized to the corresponding N-oxide, S-oxide, or S,S-dioxide. Non-limiting examples of monocyclic heterocycloalkyls include diazapanyl, piperidinyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, lactam, and lactone, and the like.

In the present specification, “cycloalkenyl” means a monocyclic or polycyclic non-aromatic ring system including at least one carbon-carbon double bond. This cycloalkenyl can be unsubstituted or substituted by one or more substituents which may be the same or different. Non-limiting examples of monocyclic cycloalkenyls include cyclopentenyl, cyclohexenyl, and cyclohepta-1,3-dienyl. Non-limiting examples of polycyclic cycloalkenyls include norbornylenyl. This cycloalkenyl may also be a heterocycloalkenyl including one or more hetero atoms (for example, an oxygen atom, nitrogen atom, or sulfur atom) as ring constituent atoms, and nitrogen atoms or sulfur atoms in the heterocycloalkenyl ring may be oxidized to the corresponding N-oxide, S-oxide, or S,S-dioxide.

In the present specification, “alkylene” is a divalent group composed of a linear or branched saturated hydrocarbon. Examples include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, 2,2-di-n-propyltrimethylene, and the like.

In the present specification, “aryl” may be either a monocyclic or fused polycyclic aromatic hydrocarbon group, or an aromatic heterocyclic group including one or more hetero atoms (for example, an oxygen atom, nitrogen atom, or sulfur atom) as ring constituent atoms. In this case, it is also sometimes called “heteroaryl” or “heteroaromatic.” When an aryl is monocyclic or a fused ring, the aryl can bond at all possible positions. Non-limiting examples of monocyclic aryls include a phenyl group (Ph), thienyl group (2- or 3-thienyl group), pyridyl group, furyl group, thiazolyl group, oxazolyl group, pyrazolyl group, 2-pyrazinyl group, pyrimidinyl group, pyrrolyl group, imidazolyl group, pyridazinyl group, 3-isothiazolyl group, 3-isooxazolyl group, 1,2,4-oxadiazol-5-yl group, or 1,2,4-oxadiazol-3-yl group. Non-limiting examples of fused polycyclic aryls include a 1-naphthyl group, 2-naphthyl group, 1-indenyl group, 2-indenyl group, 2,3-dihydroinden-1-yl group, 2,3-dihydroinden-2-yl group, 2-anthryl group, indazolyl group, quinolyl group, isoquinolyl group, 1,2-dihydroisoquinolyl group, 1,2,3,4-tetrahydroisoquinolyl group, indolyl group, isoindolyl group, phthalazinyl group, quinoxalinyl group, benzofuranyl group, 2,3-dihydrobenzofuran-1-yl group, 2,3-dihydrobenzofuran-2-yl group, 2,3-dihydrobenzothiophen-1-yl group, 2,3-dihydrobenzothiophen-2-yl group, benzothiazolyl group, benzimidazolyl group, fluorenyl group, or thioxanthenyl group. In the present specification, an aryl group may have one or more optional substituents on its ring. Examples of substituents include, but are not limited to, an alkoxy group, halogen atom, amino group, mono- or di-substituted amino group, substituted silyl group, acyl group, or the like. When an aryl group has two or more substituents, they may be the same or different. The same is also true for the aryl moiety of other substituents (for example, an aryloxy group, arylalkyl group, or the like) including an aryl moiety.

In the present specification, “alkoxy group” is a structure in which the above alkyl group is bonded to an oxygen atom. Examples include saturated alkoxy groups having a linear, branched, or cyclic configuration or a combination of such configurations. For example, a methoxy group, ethoxy group, n-propoxy group, isopropoxy group, cyclopropoxy group, n-butoxy group, isobutoxy group, s-butoxy group, t-butoxy group, cyclobutoxy group, cyclopropylmethoxy group, n-pentyloxy group, cyclopentyloxy group, cyclopropylethyloxy group, cyclobutylmethyloxy group, n-hexyloxy group, cyclohexyloxy group, cyclopropylpropyloxy group, cyclobutylethyloxy group, or cyclopentylmethyloxy group can be given as suitable examples.

The term “amide” used in the present specification includes both RNR′CO— (when R=alkyl, alkaminocarbonyl-) and RCONR′— (when R=alkyl, alkylcarbonylamino-).

The term “ester” used in the present specification includes both ROCO— (when R=alkyl, alkoxycarbonyl-) and RCOO— (when R=alkyl, alkylcarbonyloxy-).

In the present specification, the term “ring structure” means a heterocyclic or carbocyclic group when formed by a combination of two substituents. Such rings may be saturated, unsaturated, or aromatic. Therefore, the term “ring structure” includes the cycloalkyls, cycloalkenyls, aryls, and heteroaryls defined above.

In the present specification, the phrase “heterocyclic structure” is synonymous with “heterocycle” and means a monocyclic heterocycle having one or more hetero atoms selected from any of O, S, and N in the ring; such a ring can be saturated, unsaturated, or aromatic. Also, these monocyclic heterocycles can also include, for example, a ring (polycyclic heterocycle) in which one or two 3- to 8-membered rings are fused. Examples of non-aromatic heterocycles include a piperidine ring, piperazine ring, morpholine ring, and the like. In addition, examples of aromatic heterocycles include a pyridine ring, pyrimidine ring, pyrrole ring, imidazole ring, and the like. Other examples also include julolidine, indoline, and the like.

In the present specification, specific substituents can form ring structures with other substituents, and those skilled in the art can understand that a specific substitution, for example, bonding to hydrogen, is formed when such substituents bond to each other. Therefore, when it is stated that specific substituents together form a ring structure, those skilled in the art can understand that this ring structure can be formed by an ordinary chemical reaction or is generated easily. Any such ring structures and their formation processes are within the purview of those skilled in the art. In addition, the heterocyclic structure may have optional substituents on the ring.

### 2. Fluorescent Probe for Detection of Peptidase Activity of the Present Invention

The fluorescent probe for detection of peptidase activity of the present invention, in one embodiment, includes a compound having a structure represented by formula (I).

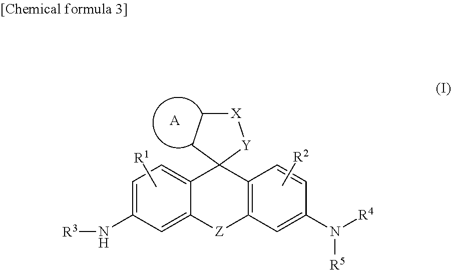

In formula (I), A represents a ring structure selected from the group consisting of a thiophene ring, a cyclopentene ring, a cyclopentadiene ring, and a furan ring. The reversibility of spirocyclization (spirocyclization equilibrium constant: pK_{cycl}) during the fluorescence response discussed later can be optimized by selecting an appropriate ring structure as said A. Preferably, A is a thiophene ring.

Ring structure A may be substituted by one or more optional substituents. Examples of such substituents include, but are not limited to, an alkyl group, alkoxy group, halogen atom, amino group, mono- or di-substituted amino group, substituted silyl group, or acyl group. These substituents may also be substituted by one or more substituents. Such substituents may have, for example, one or more alkyl groups, alkoxy groups, halogen atoms, hydroxyl groups, carboxyl groups, amino groups, sulfo groups, and the like. When A has two or more substituents on the ring, these substituents may be the same or different.

X represents a C₀-C₃ alkylene group. Said alkylene group may be substituted by a halogen atom or a haloalkyl. In the case of a C₀ alkylene group, Y means a direct bond. The alkylene group may be a linear alkylene group or a branched alkylene group. An alkylene group may be a linear alkylene group or a branched alkylene group. For example, in addition to a methylene group (—CH2-), ethylene group (—CH2-CH2-), propylene group (—CH2-CH2-CH2-), —CH(CH3)-, —CH2-CH(CH3)-, —CH(CH2CH3)-, and the like can also be used as branched alkylene groups. Among these, a methylene group, —CH(CH₃)—, or ethylene group is preferred, and a methylene group, —CH(CH₃)—, is more preferred.

Y represents O, S, C(═O), or NH. Y is preferably O. Since the Y is a site involved in the spirocyclization equilibrium constant (pK_{cycl}) in terms of the ease of the spirocyclic ring-opening reaction, the spirocyclization equilibrium constant can be adjusted by selecting an optimum Y by combination with structures such as A above.

Z represents O, C(R^{a}) (R^{b}), Si(R^{a}) (R^{b}), Ge(R^{a}) (R^{b}), Sn(R^{a}) (R^{b}), Se, P(R^{c}), or P(R^{c}) (═O). Z is preferably Si(R^{a}) (R^{b}) or C(R^{a}) (R^{b}). Here, R^{a} and R^{b} each independently represent a hydrogen atom or an alkyl group; R^{c} represents a hydrogen atom, an alkyl group, or an aryl group. When R^{a} and R^{b} are alkyl groups, the alkyl groups can have one or more substituents, and such substituents may be, for example, one or more alkyl groups, alkoxy groups, halogen atoms, hydroxyl groups, carboxyl groups, amino groups, sulfo groups, or the like. R^{a} and R^{b} are preferably both C₁-C₄ alkyl groups; more preferably, both are methyl groups (in this case, X becomes Si(CH₃)₂). In addition, in some cases, R^{a} and R^{b} may bond to each other to form a ring structure. For example, when both R^{a} and R^{b} are alkyl groups, R^{a} and R^{b} can bond to each other to form a spirocarbocycle. The ring formed is preferably, for example, about a 5- to 8-membered ring.

R¹ and R² each independently represent from one to three of the same or different substituents selected from the group consisting of a hydrogen atom, a hydroxyl group, a halogen atom, and an alkyl group, sulfo group, carboxyl group, ester group, amide group, and azide group each of which may be substituted. R¹ and R² preferably are both hydrogen atoms.

R³ represents an acyl residue derived from an amino acid. Here, said acyl residue means a residue which is a partial structure remaining after an OH group has been removed from a carboxyl group of an amino acid. Specifically, the carbonyl moiety of the acyl residue derived from an amino acid and the NH adjacent to R³ in formula (I) form an amide bond, thereby linking with the rhodamine skeleton.

In the present specification, any compound can be used as an “amino acid” as long as it is a compound having both an amino group and a carboxyl group, including natural and non-natural compounds. The amino acid may be any of a neutral amino acid, a basic amino acid, or an acidic amino acid. In addition to amino acids that themselves function as transmitters such as neurotransmitters, amino acids that are structural components of polypeptide compounds such as bioactive peptides (including oligopeptides as well as dipeptides, tripeptides, and tetrapeptides) and proteins can be used and may be, for example, an α amino acid, β amino acid, γ amino acid, or the like. It is preferable to use an optically active amino acid as the amino acid. For example, either a D- or L-amino acid may be used for α amino acids, but it is sometimes preferable to select an optically active amino acid that functions in the body.

As discussed later, R³ is a site cleaved by a reaction with a peptidase that serves as a target. The target peptidase can be γ-glutamyl transpeptidase (GGT), dipeptidyl peptidase IV (DPP-IV), or calpain. Furthermore, when the target peptidase is γ-glutamyl transpeptidase, R³ is preferably a γ-glutamyl group. Also, when the target peptidase is dipeptidyl peptidase IV, R³ is preferably an acyl group including a proline residue. When the target peptidase is calpain, R³ can be, for example, an acyl group including a cysteine residue, or Suc-Leu-Leu-Val-Tyr(Suc-LLVY) or AcLM known in the art as calpain substrates can also be used.

R⁴ and R⁵ each independently represent a hydrogen atom or an alkyl group. When both R⁴ and R⁵ represent alkyl groups, the alkyl groups may be the same or different. For example, R⁴ and R⁵ each independently can be a methyl group or ethyl group. R⁴ and R⁵ are preferably both hydrogen atoms.

Here, when R⁴ and R⁵ are both alkyl groups, R⁴ and R⁵ together may form a 5- to 8-membered heterocyclic structure including the nitrogen atom to which they are bonded. Also, when R⁴ (or R⁵) is an alkyl group, R⁴ (or R⁵) together with R² may form a 5- to 8-membered heterocyclic structure including the nitrogen atom to which R⁴ (or R⁵) is bonded. The heterocyclic structure is preferably a 6-membered ring. Also, the heterocyclic structure can also include hetero atoms other than the nitrogen atom to which R⁴ and R⁵ are bonded.

Compounds of formula (Ia) and formula (Ib) below can be given as specific examples of compounds of formula (I) typical as the fluorescent probe for detection of peptidase activity of the present invention.

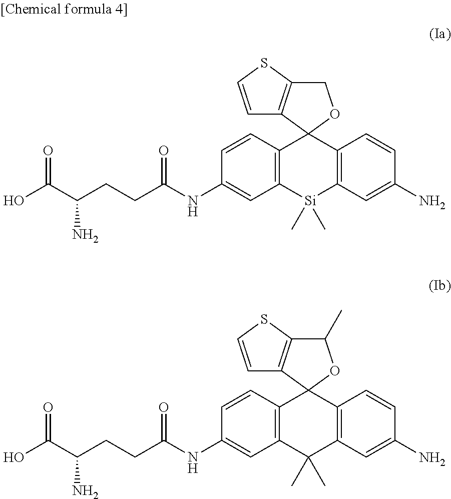 [SMILES: C[Si]1(C)c2cc(N)ccc2C2(OCc3sccc32)c2ccc(NC(=O)CC[C@H](N)C(=O)O)cc21 ; CC1OC2(c3ccc(N)cc3C(C)(C)c3cc(NC(=O)CC[C@H](N)C(=O)O)ccc32)c2ccsc21]

Compounds represented by formula (I) can exist as salts. Examples of such salts include base addition salts, acid addition salts, amino acid salts, and the like. Examples of base addition salts include sodium salts, potassium salts, calcium salts, magnesium salts, and other such metal salts, ammonium salts, or triethylamine salts, piperidine salts, morpholine salts, and other such organic amine salts. Examples of acid addition salts include hydrochlorides, sulfates, nitrates, and other such mineral acid salts, carboxylates, methanesulfonates, p-toluenesulfonates, citrates, succinates, and other such organic acid salts. Glycine salts and the like can be given as an example of amino acid salts. However, salts are not limited to these.

Compounds represented by formula (I) sometimes have one or more asymmetrical carbons in accordance with the types of substituents and can exist as stereoisomers such as optical isomers and diastereomers. Stereoisomers of a pure form, any mixtures of stereoisomers, racemic mixtures, and the like are all encompassed within the scope of the present invention.

Compounds represented by formula (I) or salts thereof can also exist as hydrates or solvates. All of these are encompassed within the scope of the present invention. The type of solvent that forms a solvate is not particularly restricted; examples include ethanol, acetone, isopropanol, and other such solvents.

The above fluorescent probe may be used as a composition by compounding with additives commonly used in the preparation of reagents as needed. For example, dissolution auxiliaries, pH adjusters, buffers, isotonifying agents, and other such additives can be used as additives for use in a physiological environment, and the amounts compounded can be selected as is appropriate by one skilled in the art. These compositions can be provided as a composition of a suitable form such as a mixture in powdered form, freeze-dried product, granules, tablets, liquid, or the like.

In addition, when peptidase activity is detected using the fluorescent probe of the present invention or when the fluorescent probe of the present invention is used for cancer diagnosis as discussed later, the fluorescent probe of the present invention can be used in the form of a kit comprising said fluorescent probe. In said kit, the fluorescent probe of the present invention is usually prepared as a solution, but the fluorescent probe of the present invention can also be provided as a composition of a suitable form such as mixture in powdered form, freeze-dried product, granules, tablets, liquid, or the like and used by being dissolved in distilled water for injection or a suitable buffer at the time of use. Said kit may also include the above additives as needed.

Since methods of producing typical compounds encompassed among compounds of the present invention represented by formula (I) are illustrated concretely in examples in this specification, any compounds encompassed by formula (I) can be produced easily by one skilled in the art by selecting the starting raw materials as needed and reagents, reaction conditions, and the like as appropriate using the disclosure of this specification as a reference.

### 3. Fluorescence Emission Mechanism of the Fluorescent Probe of the Present Invention

The fluorescence emission mechanism of the fluorescent probe for detection of peptidase activity of the present invention is explained below.

The use of formula (Ia) as compounds represented by formula (I) is illustrated. As shown on the left in the scheme below, the fluorescent probe itself is essentially non-absorbing and non-fluorescent (the fluorescence response is in the off state) at physiological pH (near pH 7.4) when the compound represented by formula (Ia) is in a state in which the upper part of the silicon rhodamine skeleton having a structure in which the central atom of the rhodamine has been substituted from O to Si is closed to form a spirocycle. In contrast, the compound on the right in the scheme in which the spirocycle moiety is opened is generated when the acyl residue derived from an amino acid of R³ (glutamic acid residue in formula (Ia)) is hydrolyzed by peptidase and cleaved from the silicon rhodamine skeleton. Said ring-opened compound exhibits strong fluorescence.

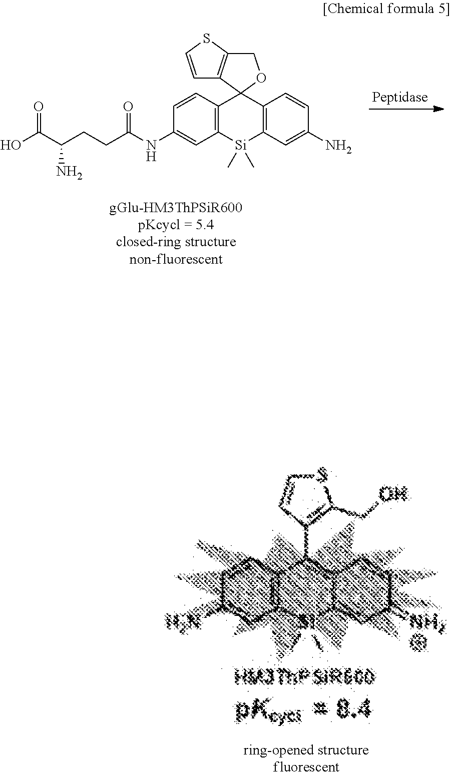 [SMILES: C[Si]1(C)c2cc(N)ccc2C2(OCc3sccc32)c2ccc(NC(=O)CC[C@H](N)C(=O)O)cc21]

Closed-ring structure, non-fluorescent, Ring-opened structure, fluorescent, peptidase

Specifically, compounds represented by formula (I), comprising formula (Ia), emit virtually no fluorescence when irradiated, for example, with excitation light of about 500-650 nm in the pH environment within the body, but ring-opened compounds generated by reaction with peptidase emit very strong fluorescence under the same conditions. Therefore, when cells that have taken up a fluorescent probe represented by formula (I) do not express a peptidase which hydrolyzes and cleaves R³, no ring-opened compound is generated and no fluorescent substance is generated within the cells. However, a ring-opened compound is generated and strong fluorescent emission is obtained when such a peptidase is present. Therefore, the presence of a peptidase that serves as the target can be detected by observing on/off changes in the fluorescence intensity and thereby detecting the presence of cancer cells or the like the express the peptidase.

Also, compounds represented by formula (I) have the feature that the fluorescence peak wavelength of the fluorescence emission due to opening of the spirocycle can be fluorescence in the red region near 600 nm by adjusting the type of Z, which is the position 10 element of the xanthene ring, and the type of cyclic structure A linked to the xanthene skeleton. This makes it possible to visualize cancer cells and the like present deep in the body, such as lymph node metastases, which was difficult to do in the past.

A feature when using the fluorescent probe of the present invention in living cells is that the ring-opened compound created by hydrolysis of the compound of formula (I) by peptidase accumulates in the lysosomes of cells. The low pH within the lysosomes shifts the spirocyclization equilibrium, changing the compound from a closed-ring structure to a ring-opened structure and obtaining a fluorescence response. The background signal emitted from probe that has leaked from the cells is suppressed, and high-sensitivity detection is possible.

### 4. Method for Detecting Peptidase Activity Using the Fluorescent Probe of the Present Invention

In accordance with the emission mechanism, target cells that express a specific peptidase can be specifically detected or visualized using the fluorescent probe of the present invention. Specifically, only target cells that express a specific peptidase can be detected or visualized as a near infrared fluorescence signal specifically by comprising A) a step for bringing the fluorescent probe and target cells into contact in vivo or ex vivo; and a step for observing a fluorescence response due to a reaction between a peptidase specifically expressed in the target cells and the fluorescent probe. Furthermore, in the present specification, the term “detection” should be interpreted in the broadest sense to include measurement for various purposes such as quantitative and qualitative.

As discussed above, the specific peptidase can preferably be γ-glutamyl transpeptidase, dipeptidyl peptidase IV (DPP-IV), or calpain. Peptidases, however, are not limited to these. The target cells are preferably cancer cells.

Also, the method of the present invention can also include observing the fluorescence response using a fluorescence imaging means. A fluorometer having a wide measurement wavelength can be used as the means for observing the fluorescence response, but the fluorescence response can also be visualized using a fluorescence imaging means that permits display as a two-dimensional image. Since the fluorescence response can be visualized two-dimensionally by using a fluorescence imaging means, it becomes possible to instantly recognize the target cells that express peptidase. Devices known in the art can be used as the fluorescence imaging device. Furthermore, the reaction of the peptidase and fluorescent probe can also be detected in some cases by the changes in the UV-visible absorption spectrum (for example, the change in absorbance at a specific absorption wavelength).

In step A) above, typical examples of the means of bringing the fluorescent probe of the present invention into contact with the peptidase expressed specifically in the target cells include adding, applying, or spraying a solution containing the fluorescent probe, but a suitable means can be selected in accordance with the application. Also, when the fluorescent probe of the present invention is applied to diagnosis or assistance in diagnosis in an animal individual or to the detection of specific cells or tissues, the means for bringing the fluorescent probe into contact with the peptidase expressed in the target cells or tissues is not particularly restricted, and administration means common in the field such as intravenous administration can be used.

The use concentration of the fluorescent probe of the present invention is not particularly restricted; a solution having a concentration of about 0.1-100 μM, for example, can be used.

In addition, light irradiation performed on target cells can be direct irradiation of light on the cells or irradiation via a wave guide (such as an optical fiber). Any light source can be used as long as the light source is capable of irradiating light including the absorption wavelength of the fluorescent probe of the present invention after undergoing enzymatic cleavage, and the light source can be selected as is appropriate to the environment and the like in which the method of the present invention is implemented.

A compound represented by general formula (I) or a salt thereof may be used without further modification as the fluorescent probe of the present invention, but may be used in the form of a composition compounded with additives commonly used in the preparation of reagents as needed. For example, dissolution auxiliaries, pH adjusters, buffers, isotonifying agents, and other such additives can be used as additives for use in a physiological environment, and the amounts compounded can be selected as is appropriate by one skilled in the art. These compositions are generally provided as a composition of a suitable form such as a mixture in powdered form, freeze-dried product, granules, tablets, liquid, or the like, but can be used dissolved in distilled water for injection or a suitable buffer at the time of use.

When the target cells in step B) above are cancer cells or cancer tissues that express a specific peptidase, the cancer cells and cancer tissues can be detected/visualized by the detection method of the present invention. Specifically, the fluorescent probe of the present invention, and the kit and detection method comprising the same (referred to hereinafter as the “detection method of the present invention”) can also be used in the diagnosis of cancer.

In the present specification, the term “cancer tissue” means any tissue comprising cancer cells. The term “tissue” must be interpreted in the broadest sense, comprising all or part of an organ, and must not be interpreted restrictively in any sense. Since the composition for cancer diagnosis of the present invention acts to detect the peptidase strongly expressed specifically in cancer tissues, typically γ-glutamyl transpeptidase, tissues that express a high level of γ-glutamyl transpeptidase are preferred a cancer tissue. Also, the term “diagnosis” in the present specification must be interpreted in the broadest sense, including confirmation of cancer tissue at any site in the body visually or under a microscope.

The detection method of the present invention can be used, for example, during surgery or during testing. In the present specification, the term “surgery” encompasses any surgery used for the treatment of cancer, including endoscopic surgery such as gastroscopy, colonoscopy, laparoscopy, thoracoscopy, and the like in addition to craniotomy with fenestration, thoracotomy, or laparotomy, or skin surgery, and the like. Also, the term “testing” encompasses testing carried out on tissues isolated or collected from the body in addition to testing using an endoscope such as gastroscopy or colonoscopy and processing such as the excision and collection of tissues associated with testing.

Cancers that can be diagnosed by the detection method of the present invention are not particularly restricted, and encompass any malignant tumor, including sarcoma, but use in the diagnosis of solid cancers is preferred. As one preferred embodiment, the fluorescent probe of the present invention is applied by a suitable method such as spraying, application, or injection or the like to all or part of a surgical field visually or under a microscope, and the application site can be irradiated with light of a wavelength of about 500 nm after from several tens of seconds to several minutes. When the application site contains cancer tissue, the tissue will emit fluorescence, allowing the tissue to be identified as cancer tissue and removed together with the surrounding tissue including the cancer tissue. For example, in the surgical treatment of typical carcinomata such as stomach cancer, lung cancer, breast cancer, colon cancer, liver cancer, gall bladder cancer, pancreatic cancer, and the like, in addition to making a definitive diagnosis of cancerous tissue that can be confirmed visually, infiltration and metastasis to lymph tissues such as lymph nodes and surrounding organs and tissues is possible, and it becomes possible to determine the resection range by performing intraoperative rapid diagnosis.

Also, as another preferred embodiment, the fluorescent probe of the present invention is applied by a suitable method such as spraying, application or injection to a testing site, for example, in gastroscopy or colonoscopy. The application site is irradiated with light of a wavelength of about 500 nm after from several tens of seconds to several minutes, and if tissue that emits fluorescence is confirmed, that tissue can be identified as cancer tissue. When cancer tissue can be confirmed in endoscopy, the tissue can also be removed for testing or therapeutically excised.

The fluorescent probe and kit of the present invention may include the additives discussed above commonly used in the preparation of reagents as needed.

### 6. Device Using the Fluorescent Probe of the Present Invention

In another embodiment, the present invention also relates to a device equipped with a fluorescent probe comprising a compound of formula (1) and a fluorescence imaging means for observing a fluorescence response due to a reaction with a peptidase expressed specifically in the target cells.

Preferably, the device can be an endoscope or an in vivo fluorescence imaging device. Devices known in the art can serve as references regarding the structure of such an endoscope or fluorescence imaging device.

### EXAMPLES

The present invention will be described in further detail below using examples, but the present invention is not limited by these examples.

### [Reagents, Instruments, Etc.]

All of the organic solvents used in the reactions shown below were of dehydration grade. Commercial raw materials were purchased from the reagent manufacturers (Aldrich Chemical Co., Ltd., Tokyo Chemical Industry Co., Ltd., Wako Pure Chemical Industries, Ltd., and Dojindo Laboratories).

NMR measurement was conducted using JEOL JNM-LA300 (300 MHz for ¹H NMR, 75 MHz for ¹³C NMR) or JEOL JNM-LA400 (400 MHz for ¹H NMR, 100 MHz for ¹³C NMR). Mass spectrometry measurement was conducted using a MicrOTOF (ESI-TOF, Bruker Co., Ltd.). Sodium formate was used as an external standard during high-resolution MS (HRMS) measurement.

The HPLC instrument was a Jasco PU-1587S equipped with an Inertsil ODS-3 (10.0 mm×250 mm) reverse-phase column chromatograph (GL Science Inc.). In separation and purification, the following solvents A and B were used unless specified otherwise, and purification was carried out by mixing these solvents in any compositions.

A: distilled water (containing 0.1% trifluoroacetic acid)

B: acetonitrile (containing 20% purified water)

### Example 1

### 1. Synthesis of Fluorescent Probe

1-1. Synthesis of gGlu-MHM4ThPCR550

A fluorescent probe 1 (gGlu-MHM4ThPCR550) having the following structure which is a compound of formula (I) of the present invention was synthesized.

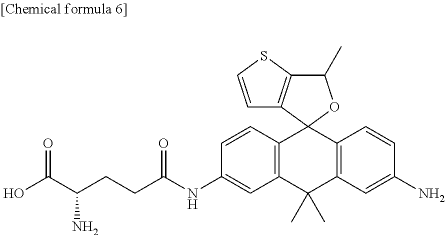 [SMILES: CC1OC2(c3ccc(N)cc3C(C)(C)c3cc(NC(=O)CC[C@H](N)C(=O)O)ccc32)c2ccsc21]

gGlu-MHM4ThPCR550 (compound 16) was synthesized according to the synthesis scheme shown below.

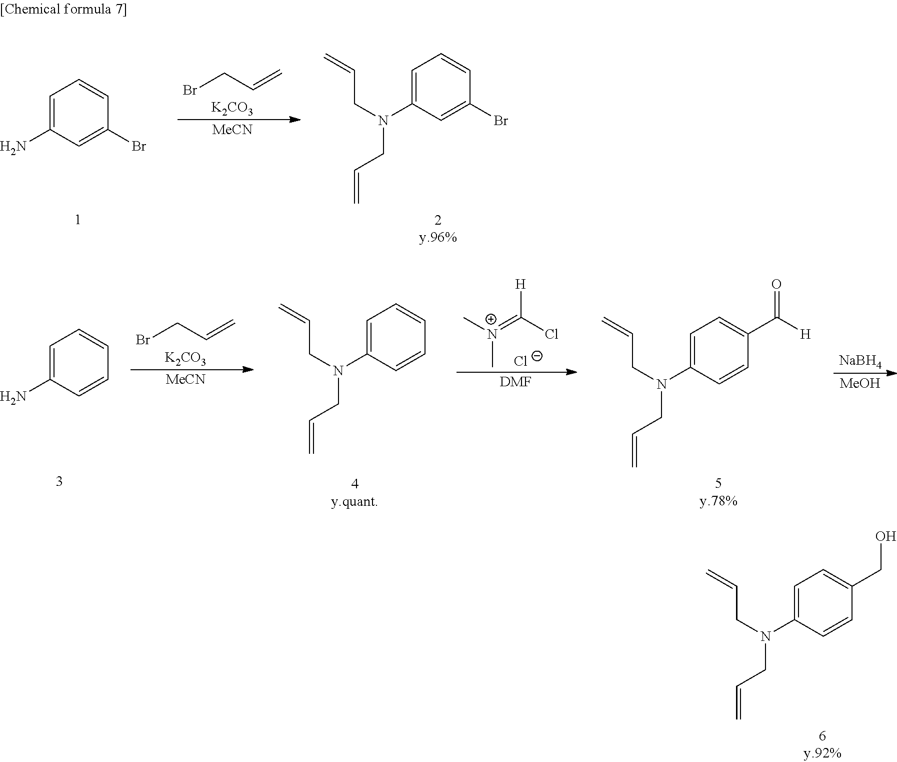

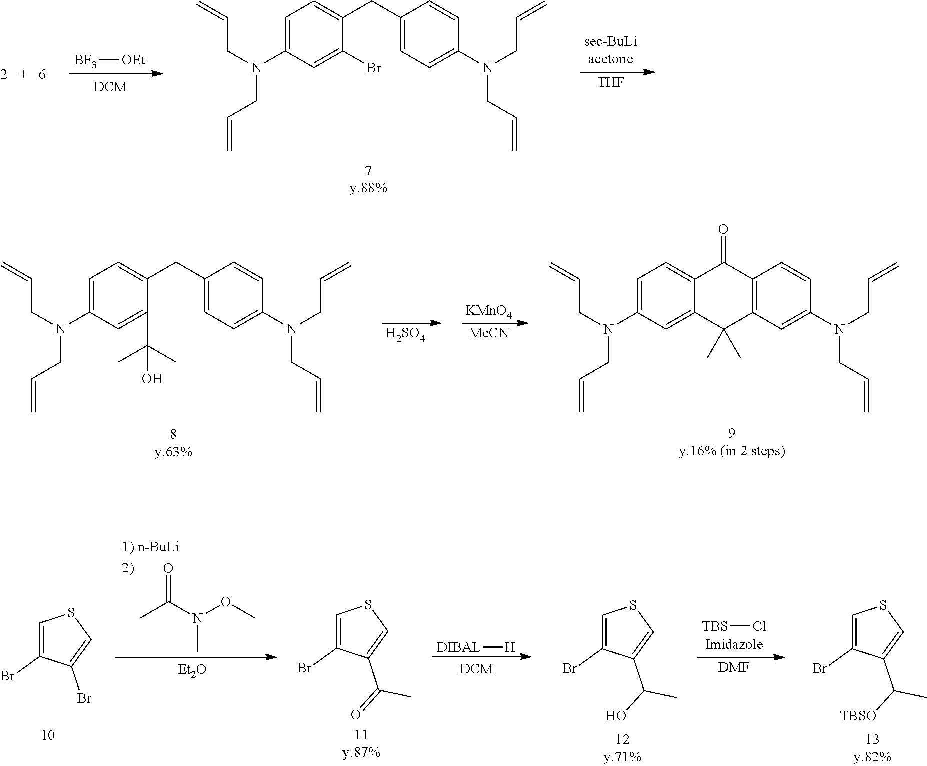

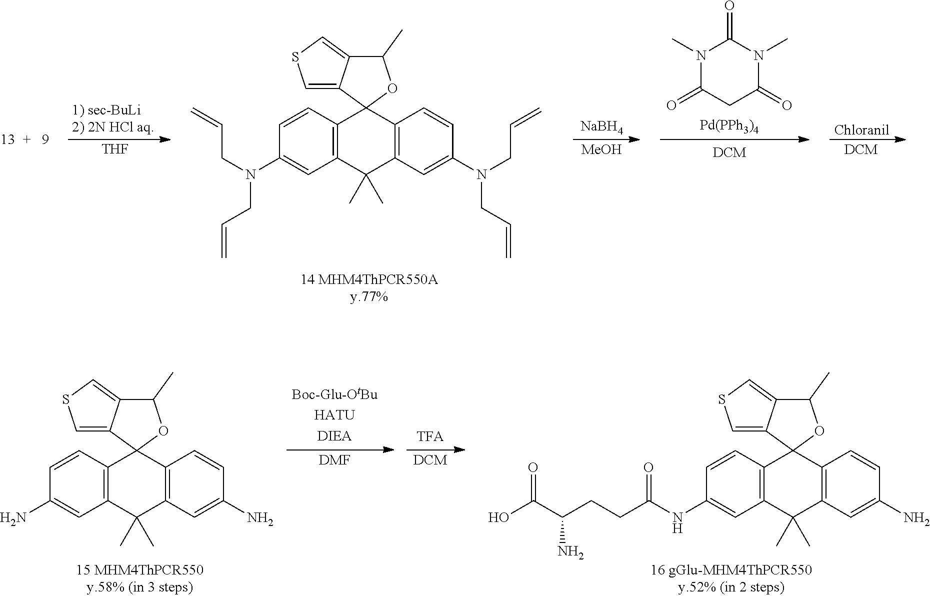

### [Synthesis of Compound 4]

Compound 4 was synthesized according to the literature (O'Sullivan, S., Doni, E., Tuttle, T. and Murphy, J. A., Angew. Chem., 2014, 53, 474-478).

### [Synthesis of Compound 5]

Vilsmeier reagent (7.4 g, 57.7 mmol) was dissolved in anhydrous DMF (40 mL), and the mixture was stirred in an Ar atmosphere at 0° C. Next, compound 4 (10.0 g, 10.9 mL, 57.7 mmol) was added, and stirring was continued for 20 hours at room temperature. Saturated NaHCO₃ was added to terminate the reaction, and the mixture was extracted using CH₂Cl₂. The organic solution was dried using Na₂SO₄, filtered, and evaporated. The residue was purified by flash column chromatography (silica gel, n-hexane/AcOEt=9/1 to 2/1), and colorless, liquid compound 5 was obtained (9.14 g, 79%).

¹H NMR (400 MHz, CDCl₃): δ3.99 (d, 4H, J=5.6 Hz), 5.12-5.20 (m, 4H), 5.79-5.87 (m, 2H), 6.69 (d, 2H, J=9.2 Hz), 7.69 (d, 2H, J=9.2 Hz), 9.71 (s, 1H).

¹³C NMR (100 MHz, CDCl₃): δ52.8, 111.5, 116.8, 125.7, 132.1, 132.3, 153.3, 190.3.

### [Synthesis of Compound 6]

Compound 5 (8000 mg, 39.7 mmol) was dissolved in anhydrous methanol (50 mL) and stirred at 0° C. Sodium tetrahydroborate (1654 mg, 43.7 mmol) was added, and stirring was continued for 4 hours at room temperature. H₂O was added to terminate the reaction, and the mixture was extracted using CH₂Cl₂. The organic solution was dried using Na₂SO₄, filtered, and evaporated. The residue was purified by flash column chromatography (silica gel, n-hexane/AcOEt=2/1 to 1/1), and colorless, liquid compound 6 was obtained (7450 mg, 92%).

¹H NMR (400 MHz, CDCl₃): 53.92 (d, 4H, J=4.0 Hz), 4.53 (s, 2H), 5.14-5.19 (m, 4H), 5.80-5.89 (m, 2H), 6.67 (d, 2H, J=9.2 Hz), 7.19 (d, 2H, J=9.2 Hz).

¹³C NMR (100 MHz, CDCl₃): δ52.9, 65.4, 112.4, 116.1, 128.7, 128.8, 133.9, 148.5.

HRMS (ESI⁺): Calcd for [M+H]⁺, 204.13884, Found, 204.13520 (−3.64 mmu).

### [Synthesis of Compound 7]

Compound 2 (2522 mg, 10.0 mmol) and compound 6 (2030 mg, 10.0 mmol) were dissolved in anhydrous CH₂Cl₂ (20 mL) and stirred at 0° C. A boron trifluoride-ethyl ether complex (2.5 mL, 20.0 mmol) was added, and stirring was continued for 22 hours at room temperature. The reaction was terminated using saturated NaHCO₃ aqueous solution, and the mixture was extracted using CH₂Cl₂. The organic solution was dried using Na₂SO₄, filtered, and evaporated. The residue was purified by flash column chromatography (silica gel, n-hexane/AcOEt=10/0 to 8.2), and colorless, liquid compound 7 was obtained (3870 mg, 88%).

¹H NMR (400 MHz, CDCl₃): δ3.85-3.90 (m, 10H), 5.12-5.19 (m, 8H), 5.77-5.90 (m, 4H), 6.55 (dd, 1H, J=8.4 Hz, 2.8 Hz), 6.63 (d, 2H, J=8.4 Hz), 6.87 (d, 1H, J=2.8 Hz), 6.93 (d, 1H, J=8.4 Hz), 7.02 (d, 2H, J=8.4 Hz).

¹³C NMR (100 MHz, CDCl₃): δ39.7, 52.8, 52.9, 111.8, 112.5, 116.0, 116.1, 116.3, 125.5, 128.4, 128.6, 129.6, 131.1, 133.6, 134.4, 147.1, 148.1

HRMS (ESI⁺): Calcd for [M+H]⁺, 437.15924, 439.15719, Found, 437.16055, 439.15909 (+1.32 mmu, +1.90 mmu).

### [Synthesis of Compound 8]

Compound 7 (1800 mg, 4.1 mmol) and anhydrous THF (15 mL) were added to a dry flask filled with Ar. The mixture was cooled to −78° C., 1 M sec-BuLi (4.1 mL, 4.1 mmol) was added, and acetone (0.6 mL, 8.2 mmol) was also added. The mixture was stirred for 3 hours at room temperature. H₂O was added and the reaction was terminated, and the mixture was extracted from a saturated NaHCO₃ aqueous solution using CH₂Cl₂. The organic solution was dried using Na₂SO₄, filtered, and evaporated. The residue was purified by flash column chromatography (silica gel, n-hexane/AcOEt=10/0 to 8/2), and colorless, solid compound 8 was obtained (1073 mg, 63%).

¹H NMR (400 MHz, CDCl₃): δ1.60 (s, 6H), 1.76 (s, 1H), 3.87-3.91 (m, 8H), 4.16 (s, 2H), 5.11-5.22 (m, 8H), 5.79-5.92 (m, 4H), 6.56 (d, 1H, J=8.0 Hz), 6.61 (d, 2H, J=7.2 Hz), 6.82 (s, 1H), 6.93-6.97 (m, 3H).

¹³C NMR (75 MHz, CDCl₃): δ31.8, 38.0, 53.0, 53.2, 74.3, 110.2, 111.3, 112.6, 116.0, 116.2, 126.5, 129.4, 130.9, 134.0, 134.4, 134.6, 146.6, 146.9, 146.9.

### [Synthesis of Compound 9]

Compound 8 (8900 mg, 21.4 mmol) was dissolved in 95% H₂SO₄ (10 mL) and stirred for 10 minutes at 0° C. A saturated NaHCO₃ aqueous solution was added and the reaction was terminated, and the mixture was extracted using CH₂Cl₂. The organic solution was dried using Na₂SO₄, filtered, and evaporated. Thereafter, the residue was dissolved in acetonitrile (120 mL) and stirred at 0° C. KMnO₄ (10,128 mg, 64.1 mmol) was added in small amounts. The mixture was stirred for 2 hours at room temperature, and methanol was added and the reaction was terminated. The mixture was filtered by Celite and evaporated. Light yellow, solid compound 9 (1420 mg, 16%) was obtained by purifying the residue by flash column chromatography (silica gel, CH₂Cl₂/methanol=100/0 to 97/3).

¹H NMR (400 MHz, CDCl₃): δ1.63 (s, 6H), 4.02 (d, 8H, J=2.8 Hz), 5.20-5.23 (m, 8H), 5.84-5.93 (m, 4H), 6.72 (dd, 2H, J=2.0 Hz, 8.8 Hz), 6.76 (d, 2H, J=2.0 Hz), 8.20 (d, 2H, J=8.8 Hz).

¹³C NMR (100 MHz, CDCl₃): δ33.6, 38.1, 53.0, 108.5, 111.1, 116.6, 120.3, 129.2, 133.3, 151.8, 152.3, 181.1.

HRMS (ESI⁺): Calcd for [M+H]⁺, 413.25929, Found, 413.25696 (−2.23 mmu).

### [Synthesis of Compound 11]

Compound 11 was synthesized according to the literature (S. Gao, Z. Wu, F. Wu, A. Lin, H. Yao, Adv. Synth. Catal. 2016, 358, 4129)

### [Synthesis of Compound 12]

Compound 11 (1000 mg, 4.9 mmol) was dissolved in anhydrous THF (20 mL) and stirred at 0° C. Sodium tetrahydroborate (278 mg, 7.4 mmol) was added and stirred for 22 hours at room temperature. The reaction was terminated using 1N HCl aqueous solution. The mixture was extracted using CH₂Cl₂. The organic solution was dried using Na₂SO₄, filtered, and evaporated. The residue was purified by flash column chromatograph (silica gel, n-hexane/AcOEt=9/1 to 7/3), and colorless, liquid compound 12 was obtained (714 mg, 71%).

¹H NMR (400 MHz, CDCl₃): δ1.49 (d, 3H, J=6.8 Hz), 2.43 (d, 1H, J=4.4 Hz), 4.91-4.97 (m, 1H), 7.23 (d, 1H, J=3.6 Hz), 7.27 (d, 1H, J=3.6 Hz).

¹³C NMR (100 MHz, CDCl₃): δ23.4, 66.0, 109.9, 121.3, 123.7, 145.2.

### [Synthesis of Compound 13]

Compound 12 (1077 mg, 5.23 mmol), tert-butyldimethylchlorosilane (2366 mg, 15.7 mmol), and imidazole (2136 mg, 31.4 mmol) were dissolved in anhydrous DMF (12 mL). The solution was stirred for four hours at room temperature in an Ar atmosphere. The mixture was extracted from saline using n-hexane. The organic solution was dried using Na₂SO₄, filtered, and evaporated. The residue was purified by flash column chromatography (silica gel, n-hexane/AcOEt=10/0 to 9/1) to obtain colorless, liquid compound 13 (1384 mg, 82%).

¹H NMR (400 MHz, CDCl₃): δ0.01 (s, 3H), 0.06 (s, 3H), 0.90 (s, 9H), 1.41 (d, 3H, J=6.0 Hz), 4.91 (q, 1H, J=6.0 Hz), 7.20 (d, 1H, J=3.6 Hz), 7.26 (d, 1H, J=3.6 Hz).

¹³C NMR (100 MHz, CDCl₃): δ −4.86, −4.82, 18.3, 25.6, 25.9, 67.5, 108.9, 121.2, 123.1, 146.5.

### [Synthesis of Compound 14 (MHM4ThPCR550A)]

Compound 13 (546 mg, 1.7 mmol) and anhydrous THE (12 mL) were added to a dry flask filled with Ar. The mixture was cooled to −85° C., and 1 M sec-BuLi (1.6 mL, 1.7 mmol) was added. An anhydrous THF (4 mL) solution of compound 9 (140 mg, 0.34 mmol) was added thereto. The mixture was stirred for one hour at room temperature. The reaction was terminated using 2N HCl aqueous solution. The mixture was extracted from saturated NaHCO₃ aqueous solution using CH₂Cl₂. The organic solution was dried using Na2SO4, filtered, and evaporated. The residue was purified by preparative HPLC under the following conditions: A/B=80/20 (0 min)—0/100 (30 min), linear gradient (solvent A: H₂O, 0.1% TFA; solvent B: acetonitrile/H₂O=80/20, 0.1% TFA). Dark purple, solid compound 14 was obtained (137 mg, 77%).

¹H NMR (400 MHz, CD₃OD): δ1.23 (d, 3H, J=6.0 Hz), 1.69 (s, 3H), 1.74 (s, 3H), 4.28-4.32 (m, 8H), 4.46 (q, 1H, J=6.0 Hz), 5.26 (d, 4H, J=17.6 Hz), 5.28 (d, 4H, J=10.4 Hz), 5.89-5.97 (m, 4H).

¹³C NMR (100 MHz, CD₃OD): δ23.3, 32.0, 33.7, 41.6, 53.5, 64.5, 111.7, 111.7, 113.4, 113.4, 116.6, 121.3, 121.4, 122.1, 126.8, 131.4, 134.2, 137.7, 138.0, 147.4, 156.4, 156.4, 157.2, 161.8.

HRMS (ESI⁺): Calcd for [M+H]⁺, 523.27831, Found, 523.27729 (−1.02 mmu).

### Synthesis of Compound 15 (MHM4ThPCR550)

Compound 14 (125 mg, 0.24 mmol) was dissolved in methanol (20 mL) and stirred at 0° C. Sodium tetrahydroborate (18 mg, 0.48 mmol) was added, and stirring was continued for 15 minutes at room temperature. The reaction was terminated using saturated NaHCO₃ aqueous solution. The mixture was extracted using CH₂Cl₂. The organic solution was dried using Na₂SO₄, filtered, and evaporated. The residue was dissolved in dehydrated CH₂Cl₂ (20 mL), and 1,3-dimethylbarbituric acid (186 mg, 1.19 mmol) and Pd(PPh₃)₄ (58 mg, 0.05 mmol) were added. This solution was stirred for 14 hours at 35° C. in an Ar atmosphere. Next, chloranil (118 mg, 0.48 mmol) was added, and stirring was continued for 30 minutes at room temperature. The mixture was extracted from 2N NaOH aqueous solution using CH₂Cl₂. The organic solution was dried using Na2SO4, filtered, and evaporated. The residue was purified by preparative HPLC under the following conditions: eluted by A/B=80/20 (0 min) to 0/100 (60 min), linear gradient (solvent A: H₂O, 0.1% TFA; solvent B: acetonitrile/H₂O=80/20, 0.1% TFA). Purple, solid compound 15 was obtained (51 mg, 58%).

¹H NMR (400 MHz, CD₃OD): δ1.23 (d, 3H, J=6.4 Hz), 1.66 (s, 3H), 1.71 (s, 3H), 4.46 (q, 1H, J=6.4 Hz), 6.62 (dd, 2H, J=9.2 Hz, 3.2 Hz), 7.12 (d, 1H, J=3.2 Hz), 7.13 (d, 1H, J=3.2 Hz), 7.16 (d, 1H, J=9.2 Hz), 7.21 (d, 1H, J=9.2 Hz), 7.42 (d, 1H, J=3.2 Hz), 7.63 (d, 1H, J=3.2 Hz).

¹³C NMR (100 MHz, CD₃OD): δ23.4, 31.5, 33.4, 41.0, 64.6, 112.6, 112.7, 114.6, 114.6, 120.7, 120.9, 121.9, 126.5, 134.4, 138.5, 138.8, 147.4, 157.9, 159.3, 159.4, 161.4.

HRMS (ESI⁺): Calcd for [M+H]⁺, 363.15311, Found, 363.15147 (−1.64 mmu).

### Synthesis of Compound 16 (gGlu-MHM4ThPCR550)

Compound 15 (31 mg, 0.085 mmol), boc-Glu-OtBu (13 mg, 0.043 mmol), and N,N-diisopropylethylamine (110 mg, 0.85 mmol) were dissolved in anhydrous DMF (2 mL) and stirred at room temperature. HATU (16.2 mg, 0.043 mmol) was added, and stirring was continued for two hours. The mixture was evaporated, and the residue was dissolved in CH₂Cl₂ (5 mL) and trifluoracetic acid (5 mL) and stirred for one hour at 40° C. Thereafter, the mixture was evaporated. The residue was purified by preparative HPLC under the following conditions: eluted by A/B=80/20 (0 min)—0/100 (45 min), linear gradient (solvent A: H₂O, 0.1% TFA; solvent B: acetonitrile/H₂O=80/20, 0.1% TFA). Orange, solid compound 16 was obtained (11 mg, 52%).

¹H NMR (400 MHz, CD₃OD): δ1.24-1.30 (m, 3H), 1.71 (s, 3H), 1.77 (s, 3H), 2.20-2.32 (m, 2H), 2.75 (t, 2H, J=7.2 Hz), 4.05 (t, 2H, J=7.2 Hz), 4.42-4.50 (m, 1H), 6.83 (t, 1H, J=8.4 Hz), 7.20-7.34 (m, 2H), 7.42-7.52 (m, 2H), 7.59 (d, 1H, J=8.4 Hz), 7.66 (t, 1H, J=3.2 Hz), 8.26 (s, 1H).

¹³C NMR (100 MHz, CD₃OD): δ22.9, 23.3, 25.4, 31.1, 31.1, 32.2, 33.1, 41.3, 52.4, 64.4, 64.6, 115.2, 115.3, 116.9, 117.0, 117.8, 118.0, 118.1, 122.4, 122.6, 124.5, 124.6, 125.8, 125.9, 127.3, 127.3, 133.7, 134.0, 134.4, 134.8, 141.8, 142.2, 145.4, 145.4, 147.1, 147.5, 152.6, 160.8, 161.1, 161.8, 163.4, 163.5, 170.6, 171.9.

HRMS (ESI⁺): Calcd for [M+H]⁺, 492.19570, Found, 492.19380 (−1.90 mmu).

1-2. Synthesis of gGlu-MHM4ThPCR550

Fluorescent probe 2 (gGlu-HM3ThPSiR600) having the following structure, which is a compound of formula (I) of the present invention, was synthesized.

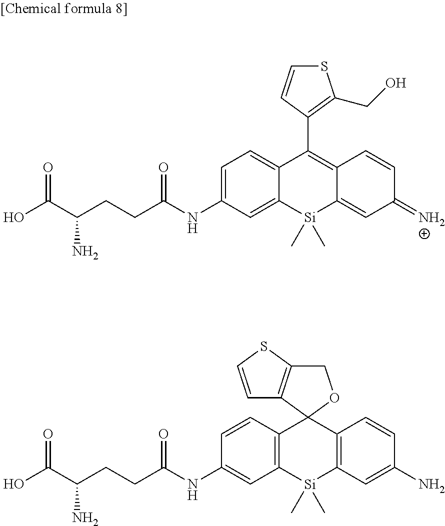

gGlu-MHM4ThPCR550 (compound 44) was synthesized by the synthesis scheme shown below.

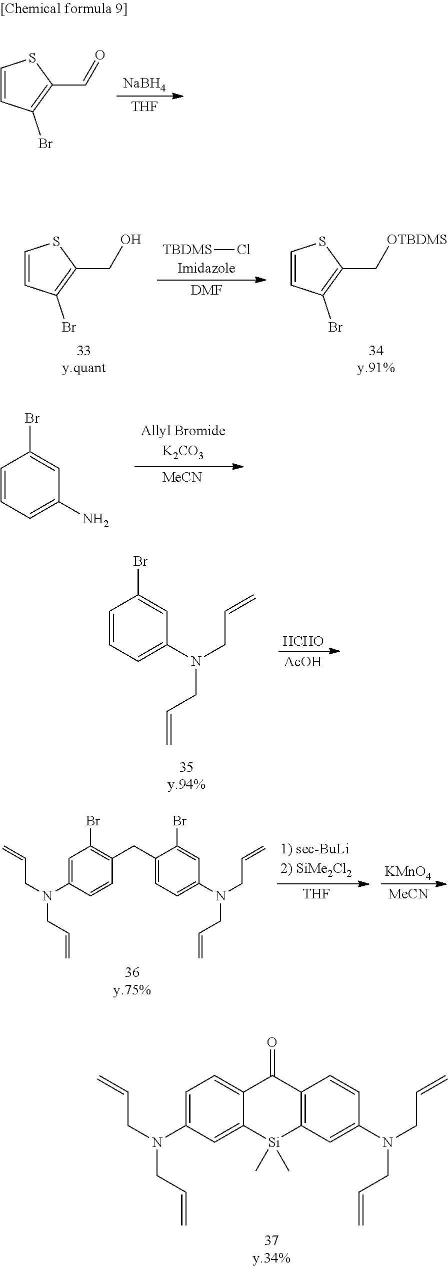

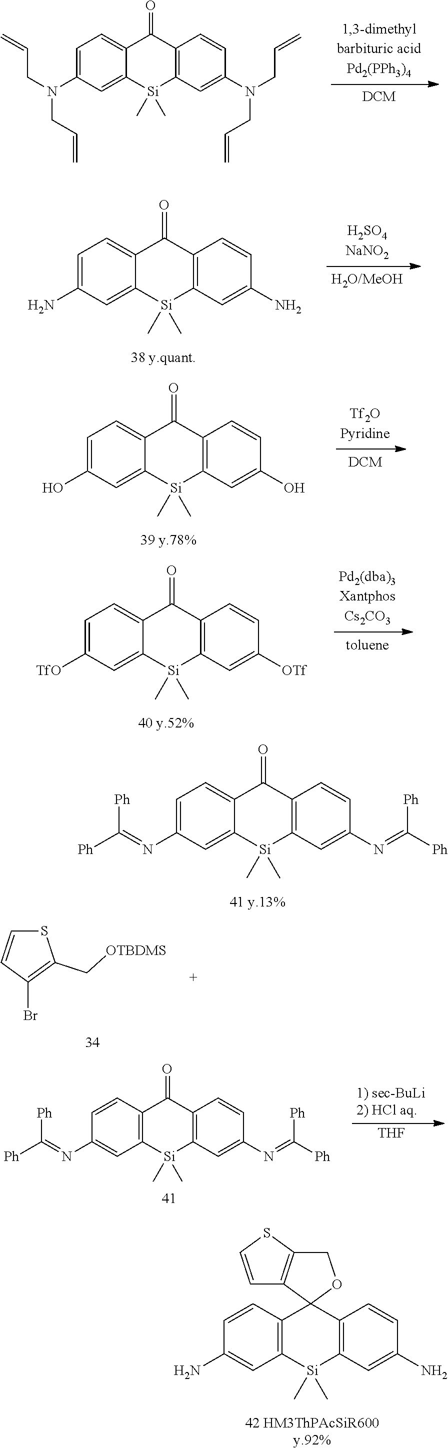

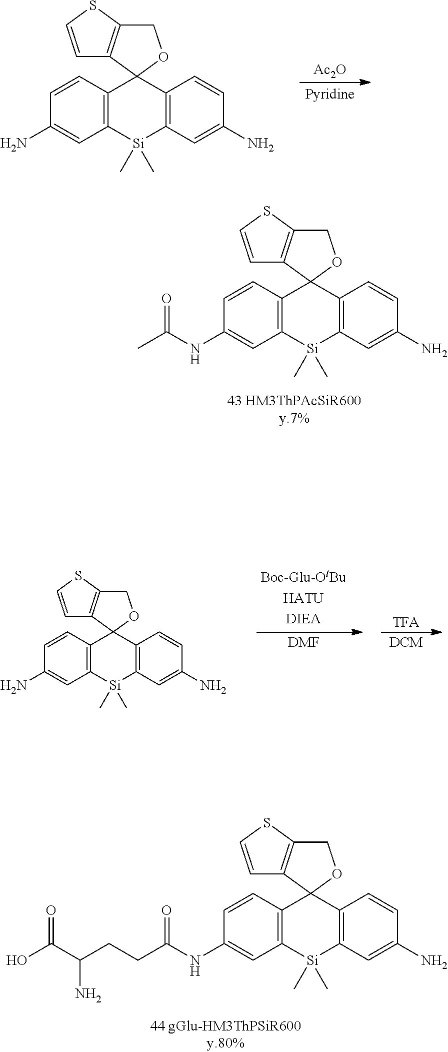

### [Synthesis of Compound 33]

3-Bromothiophene-2-carboxyaldehyde (1910 mg, 10.0 mmol) was dissolved in anhydrous THF (40 mL) and stirred at 0° C. Sodium tetrahydroborate (757 mg, 20.0 mmol) was added, and stirring was continued for three hours at room temperature. The reaction was terminated using 2N HCl. The mixture was extracted using CH₂Cl₂. The organic solution was dried using Na2SO4, filtered, and evaporated. The residue was purified by flash column chromatograph (silica gel, n-hexane/AcOEt=9/1 to 7/3), and colorless, liquid compound 33 was obtained (2015 mg, quantitative).

¹H NMR (300 MHz, CDCl₃): δ2.25 (t, 1H, J=5.1 Hz), 4.79 (d, 2H, J=5.1 Hz), 6.96 (d, 1H, J=5.9 Hz), 7.27 (d, 1H, J=5.9 Hz).

### [Synthesis of Compound 34]

Compound 33 (2000 mg, 10.36 mmol), tert-butyldimethylchlorosilane (2342 mg, 15.54 mmol), and imidazole (2116 mg, 31.08 mmol) were dissolved in anhydrous DMF (20 mL) and stirred for three hours at room temperature in an Ar atmosphere. The mixture was extracted from saline using n-hexane. The organic solution was dried using Na₂SO₄, filtered, and evaporated. The residue was purified by flash column chromatography (silica gel, n-hexane), and colorless, liquid compound 34 was obtained (2884 mg, 91%).

¹H NMR (400 MHz, CDCl₃): δ0.11 (s, 6H), 0.93 (s, 9H), 4.80 (s, 2H), 6.90 (d, 1H, J=4.8 Hz), 7.20 (d, 1H, J=4.8 Hz).

¹³C NMR (100 MHz, CDCl₃): δ−5.2, 18.4, 25.9, 60.7, 106.1, 124.6, 129.8, 140.4

### [Synthesis of Compounds 35-39]

Compounds 35-39 were synthesized according to the literature (Hirabayashi, K.; Hanaoka, K.; Takayanagi, T.; Toki, Y.; Egawa, T.; Kamiya, M.; Komatsu, T.; Ueno, T.; Terai, T.; Yoshida, K.; Uchiyama, H.; Nagano, T.; Urano, Y. Analytical chemistry 2015, 87, 9061).

### [Synthesis of Compound 40]

Compound 39 (1600 mg, 5.92 mmol) and pyridine (1.9 mL, 23.7 mmol) were dissolved in anhydrous CH₂Cl₂ (40 mL), and the mixture was stirred at 0° C. Next, trifluoromethanesulfonic anhydride (3.9 mL, 23.7 mmol) was added, and stirring was continued for four hours. The reaction was terminated using H₂O, and the mixture was extracted using CH₂Cl₂. The organic solution was dried using Na₂SO₄, filtered, and evaporated. The residue was purified by flash column chromatography (silica gel, CH₂Cl₂) to obtain colorless, solid compound 40 (1660 mg, 52%).

¹H NMR (400 MHz, CDCl₃): δ0.55 (s, 6H), 7.48 (dd, 2H, J=2.8 Hz, 8.8 Hz), 7.57 (d, 2H, J=2.8 Hz), 8.49 (d, 2H, J=8.8 Hz).

¹³C NMR (100 MHz, CDCl₃): δ−2.4, 118.8 (q, J=320 Hz), 123.2, 125.6, 132.9, 140.0, 142.2, 152.2, 184.8

### [Synthesis of Compound 41]

Compound 40 (1500 mg, 2.8 mmol), benzophenone imine (4060 mg, 22.4 mmol), Pd₂(dba)₃ (513 mg, 0.56 mmol), xantphos (324 mg, 0.56 mmol), and Cs₂CO₃ (9123 mg, 28.0 mmol) were dissolved in degassed dioxane (50 mL), and the solution was stirred for 22 hours at 100° C. in an Ar atmosphere. The mixture was extracted using CH₂Cl₂, and the organic solution was dried using Na2SO4, filtered, and evaporated. The residue was purified by flash column chromatography (silica gel, n-hexane/AcOEt=10/0 to 7/3) to obtain yellow, solid compound 41 (220 mg, 13%).

¹H NMR (400 MHz, CD₂Cl₂): δ0.13 (s, 6H), 6.82 (d, 2H, J=2.4 Hz), 6.95 (dd, 2H, J=8.4, 2.4 Hz), 7.11-7.15 (m, 3H), 7.22-7.32 (m, 6H), 7.42-7.53 (m, 7H), 7.78 (d, 4H, J=8.0 Hz), 8.20 (d, 2H, J=8.4 Hz). ¹³C NMR (100 MHz, CD₂Cl₂) δ−1.64, 123.1, 125.2, 128.4, 128.6, 129.2, 129.6, 129.8, 130.3, 130.6, 131.5, 136.3, 139.3, 140.2, 154.6, 169.2, 186.1. HRMS (ESI⁺): calcd for [M+H]⁺, 597.23621; found, 597.23370 (−2.51 mmu).

### [Synthesis of Compound 42 (HM3ThPSiR600)]

Compound 34 (412 mg, 1.34 mmol) and anhydrous THF (10 mL) were added to a dry flask filled with Ar. The mixture was cooled to −78° C., and 1 M sec-BuLi (1.3 mL, 1.30 mmol) was added. An anhydrous THF (4 mL) solution of compound 41 (80 mg, 0.13 mmol) was added. The mixture was stirred for one hour at room temperature. The reaction was terminated using 2N HCl, and the mixture was extracted from saturated NaHCO₃ aqueous solution using CH₂Cl₂. The organic solution was dried using Na₂SO₄, filtered, and evaporated. The residue was purified by preparative HPLC under the following conditions: eluted by A/B=80/20 (0 min) to 0/100 (30 min), linear gradient (solvent A: H₂O, 0.1% TFA; solvent B: acetonitrile/H₂O, 0.1% TFA). Orange, solid compound 42 was obtained (50 mg, 92%).

¹H NMR (300 MHz, CD₃OD): δ0.37 (s, 3H), 0.46 (s, 3H), 4.37 (s, 2H), 6.69 (dd, 2H, J=8.8 Hz, 2.4 Hz), 6.91 (d, 2H, J=2.4 Hz), 7.01-7.10 (m, 4H). ¹³C NMR (75 MHz, CD₃OD): δ−0.9, 0.0, 60.5, 85.0, 118.8, 119.1, 122.0, 129.8, 132.5, 136.6, 138.7, 139.4, 147.1, 147.4. HRMS (ESI⁺): Calcd for [M]⁺, 365.11438, Found, 365.11429 (−0.09 mmu).

### [Synthesis of Compound 43 (HM3ThPAcSiR600)]

Compound 42 (20 mg, 0.055 mmol) was dissolved in anhydrous pyridine (3 mL) and stirred at 0° C. in an Ar atmosphere. Acetic anhydride (5.6 mg, 0.55 mmol) in anhydrous pyridine (1 mL) was added dropwise, and stirring was continued for 24 hours. The reaction was quenched using H₂O, and the mixture was evaporated. The residue was purified by preparative HPLC under the following conditions: eluted by A/B=80/20 (0 min) to 0/100 (30 min), linear gradient (solvent A: H₂O, 0.1% TFA; solvent B: acetonitrile/H₂O, 0.1% TFA). Red, solid compound 43 was obtained (1.5 mg, 7%).

¹H NMR (400 MHz, CD₃OD): δ0.47 (s, 3H), 0.53 (s, 3H), 2.12 (s, 3H), 5.11 (s, 2H), 6.63-6.69 (m, 2H), 6.97-7.03 (m, 2H), 7.11-7.14 (m, 1H), 7.44-7.47 (m, 2H), 7.81-7.82 (m, 1H). HRMS (ESI⁺): Calcd for [M]⁺, 407.12495, Found, 407.12319 (−1.76 mmu).

[Synthesis of Compound 44 (gGlu-HM3ThPSiR600)]

Compound 42 (30 mg, 0.082 mmol), boc-Glu-OtBu (12.4 mg, 0.041 mmol), and N,N-diisopropylethylamine (106 mg, 0.82 mmol) were dissolved in anhydrous DMF (2 mL) and stirred at room temperature. HATU (15.6 mg, 0.041 mmol) was added, and stirring was continued for one hour. The mixture was evaporated, and the residue was dissolved in CH₂Cl₂ (5 mL) and trifluoroacetic acid (5 mL) and stirred for one hour at 40° C. Thereafter, the mixture was evaporated. The residue was purified by preparative HPLC under the following conditions: eluted by A/B=80/20 (0 min) to 0/100 (45 min), linear gradient (solvent A: H₂O, 0.1% TFA; solvent B: acetonitrile/H₂O=80/20, 0.1% TFA). Red, solid compound 44 was obtained (16 mg, 80%).

¹H NMR (400 MHz, CD₃OD): δ0.55 (s, 6H), 2.18-2.36 (m, 2H), 2.74-2.79 (m, 2H), 4.09 (t, 1H, J=6.4 Hz), 4.42 (s, 2H), 6.85 (d, 1H, J=8.0 Hz), 6.97 (d, 1H, J=5.2 Hz), 7.21 (d, 1H, J=8.8 Hz), 7.41 (d, 1H, J=8.0 Hz), 7.46 (s, 1H), 7.62 (d, 1H, J=5.2 Hz), 7.72 (dd, 1H, J=8.8 Hz, 2.4 Hz), 8.15 (d, 1H, J=2.4 Hz) HRMS (ESI⁺): Calcd for [M]⁺, 494.15698, Found, 494.15708 (+0.10 mmu).

### Example 2

2. Study of Red Probe Structure Based on pK_{cycl} Calculation

The structure of suitable fluorescent probe compounds capable of exhibiting fluorescence by cleavage of an acyl residue of formula (I) by a peptidase which is the target was studied based on the calculated pK_{cycl} values.

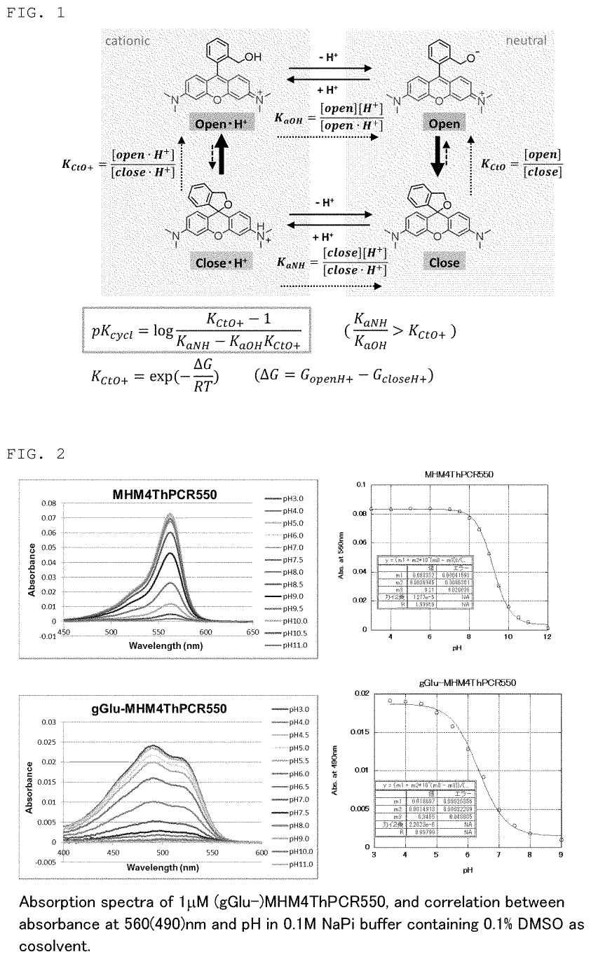

As shown in FIG. 1, an equilibrium/kinetic model consisting of only four molecular species, in consideration of protonation of amino groups and deprotonation of hydroxymethyl groups (HM), was devised as a model of intramolecular equilibrium of compounds having a rhodamine skeleton. A formula that calculates pK_{cycl} from the free energy difference of the closed-ring form/ring-opened form by the analyzed cationic reaction was derived, assuming that acid-base equilibrium (lateral direction in the model) is reached quickly enough in the HM groups and amino groups. When the calculation results of close-1, open-1 were used as this free energy difference, it was understood that pK_{cycl} of existing derivatives is accurately reproduced. Table 1 shows a comparison of the measured values and calculated results of various model structures.

TABLE 1

| 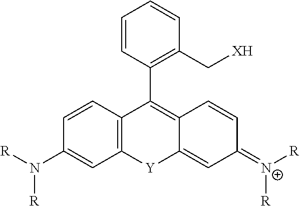 | X | Y | R | Measured | Calculated |
|---|---|---|---|---|---|
| HMRG | O | O | H | 8.1 | 7.9 |
| AMRG | NH | O | H | 6.2 | 6.2 |
| HMTMR | O | O | Me | 9.5 | 9.5 |
| AMTMR | NH | O | Me | 7.8 | 8.1 |
| HMRB | O | O | Et | 9.2 | 9.3 |
| AMRB | NH | O | Et | 8.2 | 8.1 |
| HMSIR | O | SiMe₂ | Me | 5.7 | 6.2 |
| AMSiR | NH | SiMe₂ | Me | 4.2 | 4.8 |

Measured, Calculated

Next, molecular structures having appropriate pK_{cycl} were studied using this pK_{cycl} calculation model. As an example, Table 2 shows the pK_{cycl} calculation results when using a silicon rhodamine skeleton. Here, from the viewpoint that a structure in which pK_{cycl} changes over 7.4 depending on the presence/absence of monoacetylation of the amino group (that is, the difference between SiR600 and AcSiR600) is preferable, good pK_{cycl} values were obtained for the structures shown in Table 2 especially when Ar is a thiophene ring and especially when the S atom is in position 3 viewed from the fluorophore.

TABLE 2

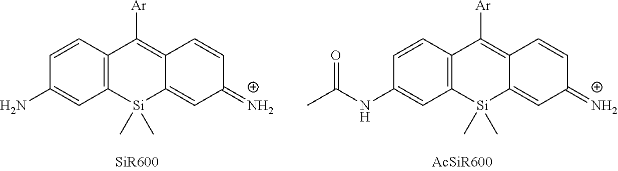

| | | pK_{cyc1} (SiR600 | pK_{cyc1} (AcSiR600 |
|---|---|---|---|
| | Ar | calculated value) | calculated value) |
| HMCPSiR600 | 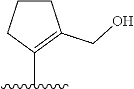 [SMILES: *C1=C(CO)CCC1] | 7.5 | 4.5 |
| HM3CPDSiR600 | 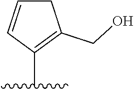 [SMILES: *C1=C(CO)CC=C1] | 7.9 | <4 |
| HM4CPDSiR600 | 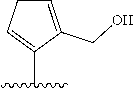 [SMILES: *C1=CCC=C1CO] | 9.8 | 5.9 |
| HM5CPDSiR600 | 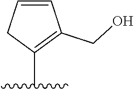 [SMILES: *C1=C(CO)C=CC1] | 8.3 | 5.9 |
| AM3FurSiR600 | 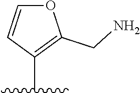 [SMILES: *c1ccoc1CN] | 11.0 | 7.7 |
| HM3ThPSiR600 | 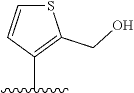 [SMILES: *c1ccsc1CO] | 8.6 | <4 |
| AM3ThPSiR600 | 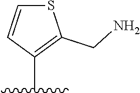 [SMILES: *c1ccsc1CN] | 5.9 | <4 |
| THNaphtSiR600 | 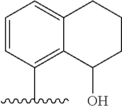 [SMILES: *c1cccc2c1C(O)CCC2] | 5.0 | — |

pK_{cyc1} (SiR600 calculated value),
pK_{cyc1} (AcSiR600 calculated value)

### Example 3

### 3. Absorption/Fluorescence Spectrum Measurement of the Fluorescent Probe of the Present Invention

The absorption spectra and fluorescence spectra of fluorescent probe 1 (gGlu-MHM4ThPCR550) and fluorescent probe 2 (gGlu-HM3ThPSiR600) synthesized in Example 1 were each measured.

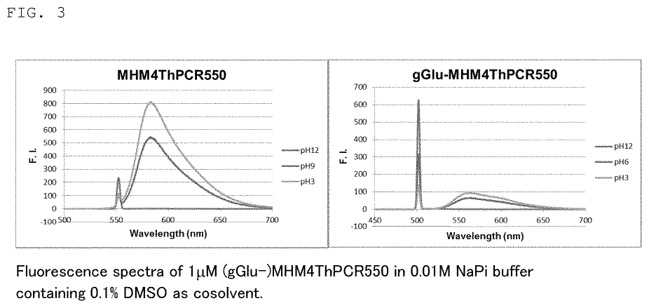

FIGS. 2 and 3 respectively show the absorption spectra and fluorescence spectra of fluorescent probe 1 (gGlu-MHM4ThPCR550) and, as a comparison, MHM4ThPCR550 having no gGlu group. The pK_{cycl} values computed from the results in FIG. 2 are shown below in Table 3.

TABLE 3

| | | | gGlu- |
|---|---|---|---|
| | MHM4ThPCR550 | MHM4ThPAcCR550 | MHM4ThPCR550 |
| pK_{cycl} | 9.2 | — | 6.3 |
| (Measured) | | | |
| pK_{cycl} | 8.7 | 5.5 | — |
| (Calculated) | | | |

In addition, as shown in FIG. 3, while the fluorescent probe of the closed-ring structure exhibited virtually no fluorescence near 660 nm, MHM4ThPCR550 which took on a ring-opened structure at pH 6 was understood to exhibit strong fluorescence intensity near 660 nm.

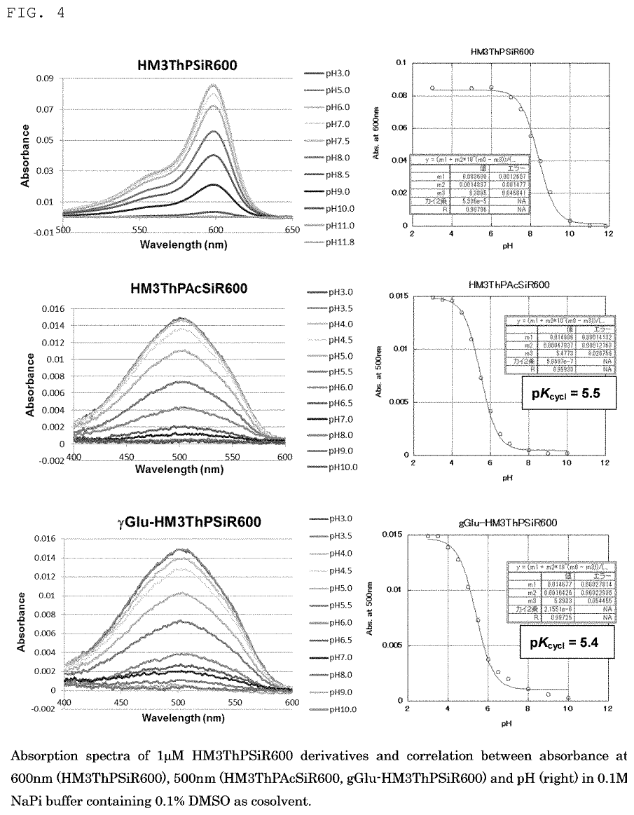

FIG. 4 shows the absorption spectrum of fluorescent probe 2 (gGlu-HM3ThPSiR600). The absorption spectra of HM3ThPSiR600 and HM3ThPAcSiR600 having no gGlu group are also shown as a comparison. Table 4 shows the pK_{cycl} values computed from the results in FIG. 4.

TABLE 4

| | | | gGlu- |
|---|---|---|---|
| | HM3ThPSiR600 | HM3ThPAcSiR600 | HM3ThPSiR600 |
| pK_{cycl} | 8.4 | 5.5 | 5.4 |
| (Measured) | | | |
| pK_{cycl} | 8.6 | <4 | — |
| (Calculated) | | | |

(Measured); Calculated

### Example 4

### 4. Enzyme Assay of Fluorescent Probes

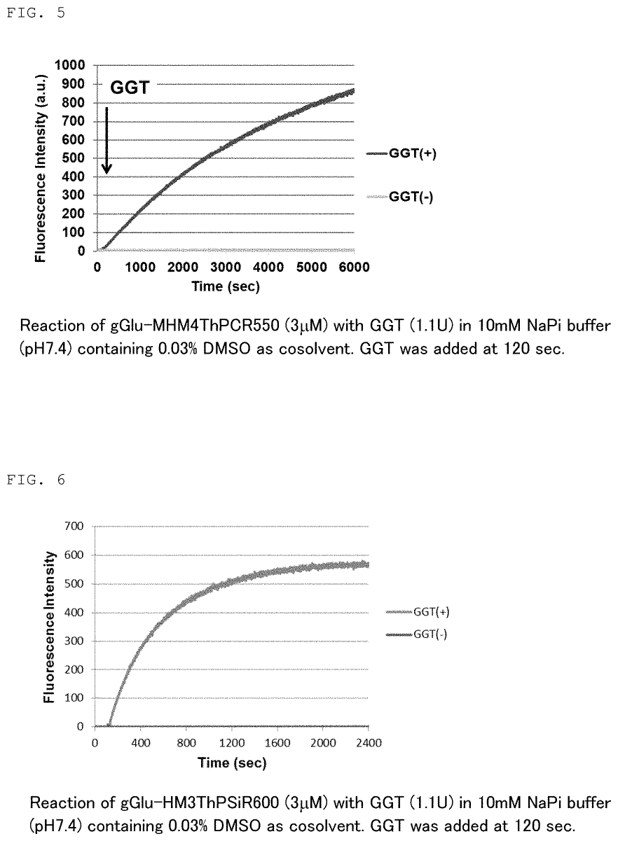

γ-Glutamyl transpeptidase (GGT) was added to fluorescent probes 1 and 2 of the present invention, and the changes in fluorescence intensity were measured. The results are shown in FIGS. 5 and 6, respectively. The arrow in FIG. 5 shows the GGT addition time.

### Experimental Conditions:

Fluorescent probe 1 or 2 was dissolved to make 1 μM in 2.5 mL of 10 mM NaPi buffer (pH 7.4) containing 0.03% DMSO. The solution, kept at 37° C., was stirred using a magnetic stirrer, and the fluorescence intensity was measured. 1.1 U of GGT was added two minutes after the start of measurement in experiments other than the negative control. The fluorescence intensity of fluorescent probe 1 at 585 nm was measured for a total of 6000 seconds and the fluorescence intensity of probe 2 at 613 nm was measured for a total of 2400 seconds, and plotted as a function of time elapsed. The excitation wavelength was 550 nm for fluorescent probe 1 and 593 nm for fluorescent probe 2; the slit width was 2.4 nm, 5.0 nm for both excitation and fluorescence, and the photomultiplier tube voltage was 700 V.

As a result, the fluorescence intensity was confirmed to increase due to addition of GGT in the case of both fluorescent probes 1 and 2. The quantum yield of fluorescent probe 1 was 0.58 at pH 3.0. The quantum yield of fluorescent probe 2 was 0.26 at pH 3.0.

### Example 5

### 5. In Vivo Imaging Using Cancer Peritoneal Dissemination Model Mice

A quantity of 300 μL of 100 μM fluorescent probes 1 and 2 was injected intraperitoneally into model mice that had been injected intraperitoneally with SHIN3. After five minutes, the mice were placed under isoflurane anesthesia and laparotomized, and imaging was conducted by a Maestro imager.

The specific experimental conditions were as follows.

Fluorescence spectrum imaging was conducted using a mouse model in which SHIN3 cells had been disseminated intraperitoneally. SHIN3-disseminated model mice were established by intraperitoneally injecting 3×10⁶ SHIN3 cells suspended in 300 μL of PBS(−) into seven-week-old female nude mice. The experiment was conducted 29-30 days after injection. The probe solution (100 μM, 300 μL) dissolved in PBS(−) was injected intraperitoneally and allowed to stand for five minutes. The mice were then anesthetized by inhalation of isoflurane, and the skin of the abdomen was cut open. The intestine was removed from the incision, placed on a black rubber plate, and the mesentery was spread. It was applied dropwise to the spread mesentery. Fluorescence imaging was conducted using a Maestro™ Ex In-Vivo Imaging System (CRi Inc.). The green-filter setting (excitation, 503 to 555 nm; emission, 580 nm long-pass) was used for fluorescent probe 1 (gGlu-MHM4ThPCR550), and the yellow-filter setting (excitation, 575 to 605 nm; emission, 645 nm long-pass) was used for fluorescent probe 2 (gGlu-HM3ThPSiR600). An image with the wavelength of fluorescence derived from the probe cut out or an image spectrally unmixed with autofluorescence is shown.

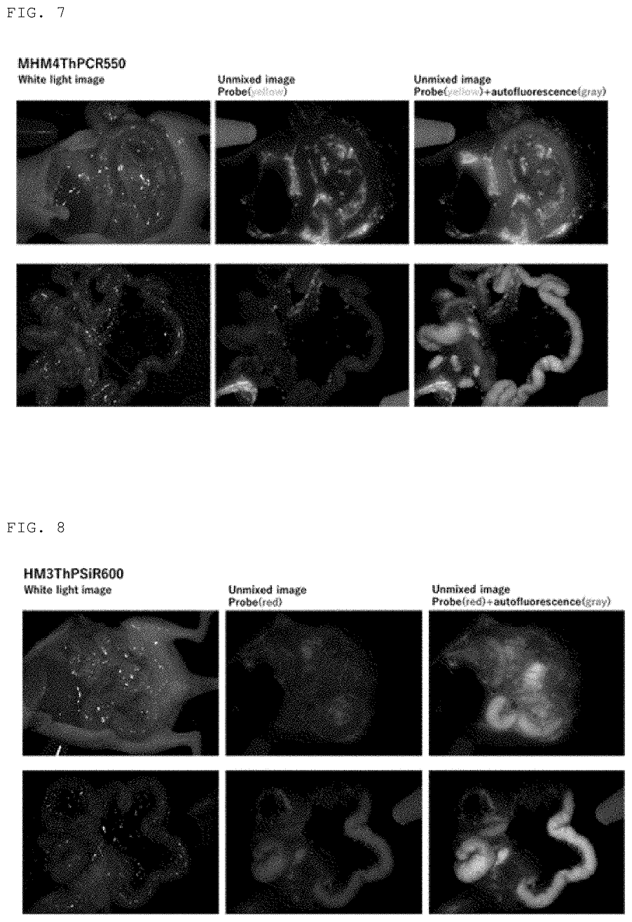

The imaging images obtained using fluorescent probes 1 and 2 are shown in FIGS. 7 and 8, respectively (upper: 200 msec; lower: 300 msec). In both cases, small tumors having adequate contrast from the background could be observed on the mesentery five minutes after administration, and the fluorescent probe of the present invention was confirmed to be capable of fluorescence imaging of microcancers on the mesentery (furthermore, the background is mainly autofluorescence from feces remaining in the intestine).

These results prove that fluorescent probes 1 and 2 of the present invention are capable of functioning as probes capable of detecting GGT and cancer cells by a red fluorescence response.

1. A compound represented by formula (I) or a salt thereof: 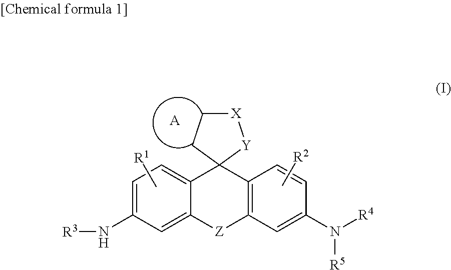
[In the formula, A represents a ring structure selected from the group consisting of a thiophene ring, a cyclopentene ring, a cyclopentadiene ring, and a furan ring;
X represents a C₀-C₃ alkylene group;
Y represents O, S, C(═O)O, or NH,
Z represents O, C(R^{a}) (R^{b}), Si(R^{a}) (R^{b}), Ge(R^{a}) (R^{b}), Sn(R^{a}) (R^{b}), Se, P(R^{c}), or P(R^{c}) (═O) (where R^{a} and R^{b} each independently represent a hydrogen atom or an alkyl group, and R^{c} represents a hydrogen atom, an alkyl group, or an aryl group);
R¹ and R² each independently represent from one to three of the same or different substituents selected from the group consisting of a hydrogen atom, a hydroxyl group, a halogen atom, and an alkyl group, sulfo group, carboxyl group, ester group, amide group, and azide group each of which may be substituted;
R³ represents an acyl residue derived from an amino acid (where the acyl residue is a residue obtained by removing an OH group from a carboxyl group of the amino acid);
R⁴ and R⁵ each independently represent a hydrogen atom or an alkyl group (where when R⁴ or R⁵ is an alkyl group, the R⁴ or R⁵, together with R², may form a ring structure comprising a nitrogen atom to which R⁴ and R⁵ are bonded).]

2. The compound or salt thereof according to claim 1, wherein A is a thiophene ring.

3. The compound or salt thereof according to claim 1, wherein Y is O.

4. The compound or salt thereof according to claim 1, wherein Z is Si(R^{a}) (R^{b}) or C(R^{a}) (R^{b}).

5. The compound or salt thereof according to claim 1, wherein R³ is a glutamic acid residue.

6. The compound or salt thereof according to claim 1, wherein R¹, R², R⁴, and R⁵ are all hydrogen atoms.

7. The compound or salt thereof according to claim 1, wherein the compound represented by formula (I) is a compound selected from the group shown below;

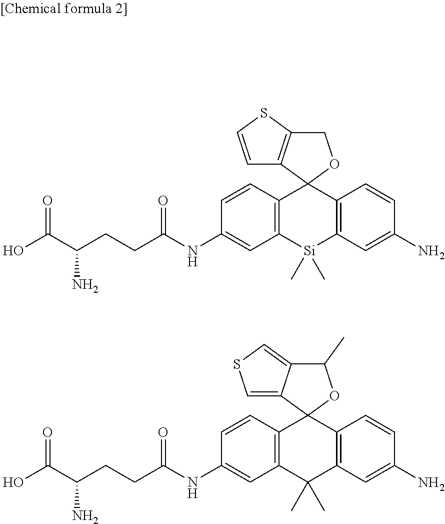 [SMILES: C[Si]1(C)c2cc(N)ccc2C2(OCc3sccc32)c2ccc(NC(=O)CC[C@H](N)C(=O)O)cc21 ; CC1OC2(c3cscc31)c1ccc(N)cc1C(C)(C)c1cc(NC(=O)CC[C@H](N)C(=O)O)ccc12]

8. A fluorescent probe for detection of peptidase activity comprising a compound or salt thereof according to any of claims 1-7.

9. A kit for detecting or for visualizing a target cell that expresses a specific peptidase comprising the fluorescent probe for detection of peptidase activity according to claim 8.

10. A kit according to claim 9, wherein the peptidase is γ-glutamyl transpeptidase, dipeptidyl peptidase IV (DPP-IV), or calpain.

11. The kit according to claim 9, wherein the target cell is a cancer cell.

12. A method for detecting or visualizing a target cell that expresses a specific peptidase using a compound or salt thereof according to any claims 1-7.

13. The method according to claim 12, characterized by comprising a step for bringing the compound or salt thereof into contact with the target cell ex vivo; and a step for observing a fluorescence response due to a reaction between a peptidase specifically expressed in the target cell and the compound or salt thereof.

14. The method according to claim 13, comprising observing the fluorescence response using a fluorescence imaging means.

15. The method according to claim 12, wherein the peptidase is γ-glutamyl transpeptidase, dipeptidyl peptidase IV (DPP-IV), or calpain.

16. The method according to claim 12, wherein the target cell is a cancer cell.

17. The use of a compound or salt thereof according to any of claims 1-7 for detecting or for visualizing a target cell that expresses a specific peptidase.

18. A device equipped with a fluorescence imaging means for observing a fluorescence response due to a reaction between a peptidase specifically expressed in a target cell and a compound or a salt thereof according any of claims 1-7.

19. The device according to claim 18, wherein the device is an endoscope or an in vivo fluorescence imaging device.
